(19) United States
(12) Patent Application Publication
(10) Pub. No.: US 2013/0071414 A1
(43) Pub. Date: Mar. 21, 2013

(54) ENGINEERED CD19-SPECIFIC T LYMPHOCYTES THAT COEXPRESS IL-15 AND AN INDUCIBLE CASPASE-9 BASED SUICIDE GENE FOR THE TREATMENT OF B-CELL MALIGNANCIES

(71) Applicants: Gianpietro Dotti, Houston, TX (US); David M. Spencer, Houston, TX (US); Cliona M. Rooney, Bellaire, TX (US); Malcolm K. Brenner, Bellaire, TX (US)

(72) Inventors: Gianpietro Dotti, Houston, TX (US); David M. Spencer, Houston, TX (US); Cliona M. Rooney, Bellaire, TX (US); Malcolm K. Brenner, Bellaire, TX (US)

(21) Appl. No.: 13/458,085

(22) Filed: Apr. 27, 2012

Related U.S. Application Data
(60) Provisional application No. 61/479,588, filed on Apr. 27, 2011.

(51) Int. Cl. A61K 39/00; C12N 5/0783; C12N 15/864; C12N 15/86; C12N 15/861; C12N 15/867; C12N 15/85; C12N 5/07; A61K 38/19

(52) U.S. Cl. CPC C12N 5/0636; C12N 15/85; C12N 5/0646; C12N 5/0638; C12N 15/86; C12N 15/861; C12N 15/867; C12N 15/8645; A61K 39/00
USPC 424/184.1; 435/320.1; 435/455; 424/93.71

(57) ABSTRACT

The present invention generally concerns particular methods and compositions for cancer therapy. In particular embodiments, there methods and compositions related to cells that harbor expression vectors encoding a cytokine and an inducible suicide gene and, optionally, the same or different vector(s) encoding a chimeric antigen receptor and/or a detectable gene product.

This application claims priority to U.S. Provisional Patent Application Ser. No. 61/479,588, filed Apr. 27, 2011, which is incorporated by reference herein in its entirety.

### STATEMENT REGARDING FEDERALLY SPONSORED RESEARCH OR DEVELOPMENT

This invention was made with government support under PO1CA94237, P50CA126752, RO1CA131027 awarded by National Institutes of Health. The government has certain rights in the invention.

### TECHNICAL FIELD

The present invention generally concerns at least the fields of cell biology, molecular biology, immunology, and medicine, such as cancer medicine, including cancer therapy and/or prevention.

### BACKGROUND OF THE INVENTION

T lymphocytes expressing a chimeric antigen receptor (CAR) can be adoptively transferred to target a range of human malignancies, including non-Hodgkin's and Hodgkin's lymphomas.¹⁻⁵ CARs most commonly combine the antigen-binding specificity of a monoclonal antibody with the effector endodomain of the CD3/T-cell receptor complex (z-chain), and redirect the specificity of T lymphocytes toward surface antigens expressed by tumor cells.⁶ CARs that target B-lineage-restricted antigens such as CD19,^{7,8} CD20⁹ and the light chain of human immunoglobulins,¹⁰ or CD30 expressed by Reed-Sternberg cells,^{2,4} have been cloned and validated in preclinical lymphoma/leukemia models, and some are currently in phase I clinical trials.^{1,3,5,11} However, it is evident from both clinical trials^{1,12,13} and preclinical models^{3,10,14} that the expansion and persistence of CAR-modified T cells in vivo are hampered by the lack of costimulatory signals after engagement with target antigens, as many tumor cells down-regulate their expression of the costimulatory molecules required for optimal and sustained T-cell function, proliferation and persistence.^{3,5}

This limitation has been partially resolved by the construction of ‘second-generation’ CARs in which a costimulatory endodomain derived from molecules such as CD28^{10,14,15} or 4-1BB^{16,17} have been incorporated within the chimeric receptors. T cells expressing these enhanced CARs retain their cytotoxic function, but, upon antigen engagement, they produce interleukin-2 (IL-2) that helps to sustain their activation and expansion,^{10,14,15} and augments antitumor activity.^{3,10,14} To further potentiate the costimulation of CAR-modified T cells, ‘third-generation’ CARs have been developed that contain multiple costimulatory endodomains such as combinations of CD28 and 4-1BB1⁸⁻²¹ or CD28 and OX40,²² which may have superior activity compared with those encoding single costimulatory endodomains.^{18-20,22} The present invention provides a solution to long-felt needs in the art

### BRIEF SUMMARY OF THE INVENTION

The present invention concerns methods and compositions for treatment of a disease condition in which the condition, including at least one symptom thereof, is improved upon exposure to polynucleotides and/or cells that harbor particular moieties. The polynucleotides and cells generally involve immunotherapy for the treatment of cancer. The compositions generally include an inducible suicide gene to destroy eventually the cells harboring the polynucleotides, a cytokine to promote proliferation of the resultant cells, and one or both of a detectable gene product and a chimeric antigen receptor that targets one or more types of cancer cells.

In some embodiments of the invention, there is an isolated mammalian cell comprising: (a) optionally a chimeric antigen receptor that targets an antigen, such as the CD19 antigen (CAR19); (b) ectopic expression of the exemplary interleukin-15 (IL-15) gene; (c) a suicide gene; and (d) optionally a detectable gene product. In certain aspects, the chimeric antigen receptor further comprises a costimulatory endodomain, such as a CD28 costimulatory endodomain, a 4-IBB costimulatory endodomain, an OX40 costimulatory endodomain, or a combination thereof. In particular embodiments of the invention, the cell comprises a polynucleotide that expresses CAR19, a polynucleotide that expresses the IL-15 gene, a polynucleotide that expresses a suicide gene, and/or a polynucleotide that expresses CAR19, IL-15, and the suicide gene. In certain embodiments, the suicide gene is caspase-9 or HSV thymidine kinase, for example.

In particular embodiments of the invention, CAR19, IL-15, suicide gene, or a combination thereof are housed on a vector, such as a plasmid or viral vector, including a retroviral vector, adenoviral vector, adeno-associated viral vector, or lentiviral vector.

In some embodiments, there are methods of improving survival, expansion and antitumor effects of CD19-specific redirected T cells, comprising the steps of transducing the CD19-specific redirected T cells with IL-15. In certain embodiments, the cells further comprise a suicide gene. In specific embodiments, a chimeric antigen receptor directed against CD19 further comprises a costimulatory endodomain.

The present invention provides an alternative strategy in which engineered CAR-modified T cells receive not only costimulation through the CD28 pathway but also ectopically produce IL-15, a cytokine crucial for T-cell homeostasis and survival.^{23,24} Embodiments also include a suicide gene that can be pharmacologically activated to eliminate transgenic cells as required^{26,27} to decrease the risk of direct toxicity and uncontrolled proliferation²⁵.

In certain embodiments of the invention, there are methods of treating an individual for cancer, comprising the step of providing to the individual a cell of the invention. Such an individual may be receiving, has received, or will receive an additional cancer therapy, such as chemotherapy, surgery, radiation, immunotherapy, hormonal therapy, or a combination thereof.

In some embodiments of the invention, there is an isolated polynucleotide, comprising a cytokine, an inducible suicide gene, and one or both of the following: a) a detectable gene product; or b) a chimeric antigen receptor. In specific embodiments, the polynucleotide is further defined as comprising a vector, such as a viral vector (adenoviral vector, a retriviral vector, a lentiviral vector, or an adeno-associated viral vector) or a plasmid. In specific embodiments, the cytokine is IL-15, IL-2, IL-7, IL-12, or IL-21. In particular embodiments, the inducible suicide gene is non-immunogenic to humans, such as caspase 9. In certain cases, the chimeric antigen receptor targets CD19, and in specific embodiments, the chimeric antigen receptor has a costimulatory endodomain from CD28, 4-IBB, OX40, or a combination thereof. In some embodiments, the detectable gene product is a nonfunctional gene product, such as ΔNGFR, a truncated form of CD19, or a truncated form of CD34, for example.

In some embodiments of the invention, there is a mammalian cell, comprising a polynucleotide as described herein. The cell is a T lymphocyte, natural killer cell, lymphokine-activated killer cell, or tumor infiltrating lymphocyte, in some embodiments.

In some embodiments of the invention, there is a method of inhibiting proliferation of a cancer cell in an individual, comprising the step of delivering to the individual a therapeutically effective amount of cells of the invention. In certain cases the method is further defined as: delivering to the individual a therapeutically effective amount of cells of the invention; releasing a relevant T cell growth factor or immunomodulating cytokine locally in the tumor microenviroment; and eliminating the cells upon exposure to the inducible gene product.

In some embodiments, there is a kit comprising a polynucleotide of the invention and/or one or more cells of the invention.

In some embodiments, there is a method of making a cell of the invention, comprising the step of introducing to the cell a polynucleotide comprising a cytokine, an inducible suicide gene, and one or both of the following: a) a detectable or selectable gene product; or b) a chimeric antigen receptor.

Other and further objects, features, and advantages would be apparent and eventually more readily understood by reading the following specification and be reference to the accompanying drawings forming a part thereof, or any examples of the presently preferred embodiments of the invention given for the purpose of the disclosure.

### BRIEF DESCRIPTION OF THE DRAWINGS

For a more complete understanding of the present invention, reference is now made to the following descriptions taken in conjunction with the accompanying drawing, in which:

FIG. 1. T cells transduced with the iC9/CAR.19/IL-15 vector produce IL-15 and expand in response to antigen stimulation. (a) The kinetics of IL-15 release by control NT, CAR.19⁺ and iC9/CAR.19/IL-15⁺ T cells with or without antigen stimulation (CD19⁺ B-CLL cells) is shown. (b) The release of IL-15 by iC9/CAR.19/IL-15⁺ T cells when these cells were maintained in culture for 4 weeks and stimulated once a week with the antigen (CD19⁺ B-CLL cells). (c) The release of IL-2 by control NT, CAR.19⁺ and iC9/CAR.19/IL-15⁺ T cells with or without antigen stimulation (CD19⁺ B-CLL cells). (d, e) The expansion of control NT, CAR.19⁺ and iC9/CAR.19/IL-15⁺ T cells upon weekly stimulation with CD19⁺ B-CLL cells. Viable cells were counted by Trypan blue exclusion once a week. Data in these panels represent the mean±s.d. of four T-cell lines.

FIG. 2. T cells transduced with the iC9/CAR.19/IL-15 vector have enhanced viability and higher expression of Bcl-2. (a) Control NT, CAR.19⁺ and iC9/CAR.19/IL-15⁺ T cells were labeled with CFSE and stimulated with CD19⁺ B-CLL cells. CFSE dilution was measured by FACS analysis after 5 days of culture on live cells. Data are representative of four T-cell lines. CFSE-negative cells of the top histogram (NT) represent residual tumor cells that are not expected to be eliminated by control T cells. (b) Annexin-V/7-AAD staining of CAR.19⁺ or iC9/CAR.19/IL-15⁺ T cells measured 5 days after the stimulation with B-CLL cells. Data are representative of four T-cell lines. (c) BCL-2 expression as detected by FACS analysis in CAR.19⁺ or iC9/CAR.19/IL-15⁺ T cells 5 days after the stimulation with B-CLL cells.

FIG. 3. In vivo localization and expansion of T cells transduced either with CAR.19 or iC9/CAR.19/IL-15 vectors. (a, d) SCID mice were infused i.v with either FFLuc-labeled Daudi or Raji cells, respectively. Tumor cell bioluminescence was measured 10 or 15 days after infusion. Then, SCID mice engrafted either with unlabeled Daudi or Raji cells, respectively, were injected either with CAR.19⁺ or iC9/CAR.19/IL-15⁺ T cells labeled with eGFP-FFLuc (b, e). T-cell signal intensity increased in mice receiving iC9/CAR.19/IL-15⁺ T cells compared with CAR.19⁺ T cells. (c, f) The maximum increase in T-cell bioluminescence obtained in 5 and 10 mice per group, respectively.

FIG. 4. iC/CAR.19/IL-15⁺ T cells have enhanced antitumor effects and lower expression of PD-1 when compared with CAR.19⁺ T cells. (a) The cytotoxic activity of control NT, CAR.19⁺ and iC9/CAR.19/IL-15⁺ T cells. Targets were CD19⁺ B-cell Lymphoma cell line (Daudi), CD19⁻ lymphoma cell line (HDLM-2) and K562 cell line. Both CAR.19⁺ and iC9/CAR.19/IL-15⁺ T cells retained specific cytotoxic activity. Data illustrate the mean±s.d. of four T-cell lines. (b) The release of interferon-γ (IFN-γ) by control, CAR.19⁺ and iC9/CAR.19/IL-15⁺ T cells with or without stimulation with the antigen (CD19⁺ B-CLL cells). Data represents the mean±s.d. of four T-cell lines. (c) The antitumor effects of CAR.19⁺ and iC9/CAR.19/IL-15⁺ cells kept in culture for 4 weeks. iC9/CAR.19/IL-15⁺ T cells had enhanced capacity to eliminate tumor cells (Karpas CD30⁺/CD19⁺) when compared with CAR.19⁺ cells. Results are representative of four T-cell lines. (d) PD-1 was significantly overexpressed in CAR.19⁺ T cells when compared with iC9/CAR.19/IL-15⁺ T cells 2 days upon stimulation with B-CLL leukemic cells.

FIG. 5. iC9/CAR.19/IL-15⁺ T cells have enhanced antitumor effects in vivo when compared with CAR.19⁺ T cells. To evaluate the antitumor effects, SCID mice were engrafted in the peritoneum (a, b) or subcutaneous (c, d) with Daudi cells labeled with FFLuc, and then treated with either control NT, CAR.19⁺ or iC9/CAR.19/IL-15⁺ T cells 7-10 days later. Tumor growth was monitored using an in vivo imaging system. (a, b) Tumor growth in representative mice. Enhanced control of tumor growth was observed in mice receiving iC9/CAR.19/IL-15⁺ T cells. (b, d) The bioluminescence signal as a measurement of tumor growth by days 38 and 24 after T-cell infusion is summarized. Enhanced control of tumor growth was observed in mice treated with iC9/CAR.19/IL-15⁺ T cells. Data represent mean±s.d. of 12 mice per group.

FIG. 6. Activation of the inducible caspase-9 suicide gene significantly eliminates iC9/CAR.19/IL-15⁺ T cells. (a) iC9/CAR.19/IL-15⁺ T cells undergo apoptosis upon incubation with CID AP20187 at 50 nM.²⁷ Results are representative of four T-cell lines. (b) SCID mice engrafted i.v. with Raji cells, infused with iC9/CAR.19/IL-15⁺ T cells expressing eGFP-FFLuc were then treated by day 14 with two doses of the CID AP20187 (50 mg) i.p. 2 days apart.²⁷ T-cell bioluminescence reduced upon CID administration. (c) The kinetics of bioluminescence in five mice before and after treatment with CID.

FIG. 7. T cells isolated from B-CLL patients and expressing iC9/CAR.19/IL-15 produce IL-15, expand in response to autologous B-CLL and provide enhanced anti-leukemia effect. (a, b) The expansion of control NT, CAR.19⁺ and iC9/CAR.19/IL-15⁺ T cells obtained from B-CLL patients upon stimulation once a week with autologous B-CLL cells. Cells were counted by Trypan blue exclusion once a week. Data in these panels represent the mean±s.d. of three T-cell lines. (c) The production of IL-2 and IL-15 by control, CAR.19⁺ and iC9/CAR.19/IL-15⁺ T cells with or without weekly stimulation with autologous CD19⁺ B-CLL cells. Data in these panels represent the mean±s.d. of three T-cell lines. (d) IL-15 protected iC9/CAR.19/IL-15⁺ T cells from apoptosis after the stimulation with B-CLL cells. Data are representative of three T-cell lines. (e) iC9/CAR.19/IL-15⁺ T cells retained enhanced capacity to eliminate autologous CD19⁺ B-CLL cells labeled with CFSE by week 4 of culture when compared with CAR.19⁺ T cells. Data are representative of three T-cell lines.

FIG. 8. Construction and expression of retroviral vectors. Panel A represents the scheme of the retroviral vectors CAR.19 and iC/CAR.19/IL-15 used to transduce activated T lymphocytes. Panel B shows the expression of the CAR.19 by transduced T cells as assessed by FACS analysis using a specific mAb recognizing the IgG1-CH2CH3 portion (spacer) of the CAR construct. CAR expression was 86%±8% (MFI 936, range 622 to 1368) for CAR.19+ T cells and 65%±7% (MFI 405, range 286 to 658) (p=0.002 for MFI comparison) for iC9/CAR.19/IL-15+ T cells indicating that the increase of cassette size from 2.0 Kb to 3.9 Kb, caused by incorporation of both the suicide gene and IL-15 within the iC9/CAR.19/IL-15 retroviral vector, only modestly decreased the expression of CAR.19.

FIG. 9. An exemplary universal construct comprising IL-15 and the inducible caspase9 gene. Panel A illustrates the schemes of the vectors. Panel B illustrates the transduction of T cells. Panel C illustrates the kinetics of IL-15 release by control NT, CAR.19⁺ and iC9/CAR.19/IL-15⁺ T cells with or without antigen stimulation (CD19⁺ B-CLL cells) (left panel).

FIG. 10. T cells transduced with the iC9/ΔNGFR/IL-15 vector produce IL-15. Panel A illustrates the scheme of the exemplary vector. Panel B illustrates the transduction of T cells. Panel C illustrates the IL-15 release by control NT and iC9/ΔNGFR/IL-15⁺ T cells with or without stimulation by immobilized OKT3 and CD28 antibodies.

### DETAILED DESCRIPTION OF THE INVENTION

As used herein, the use of the word “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method or composition described herein can be implemented with respect to any other method or composition described herein.

### I. Embodiments of Compositions of the Invention and Uses Thereof

In embodiments of the invention, there are at least nucleic acids, polypeptides, vectors, and/or cells that concern recombinantly engineered compositions having at least an inducible suicide gene and a cytokine. In addition, a chimeric antigen receptor (CAR) and/or a detectable gene product may be included in the composition. In some embodiments that include a vector, the CAR may be provided on a vector separate from a vector that harbors the inducible suicide gene and the cytokine. In embodiments for the detectable gene product, the cells that harbor the polynucleotide that encodes the detectable gene product are identifiable, such as by standard means in the art, including flow cytometry, spectrophotometry, or fluorescence, for example.

In some embodiments of the invention polynucleotides harboring the cytokine, inducible gene product, and CAR and/or detectable gene product are integrated into the genome of a mammalian cell, although in some embodiments of the invention the polynucleotides are not integrated into the genome.

As used herein, a nucleic acid construct or nucleic acid sequence or polynucleotide is intended to mean a DNA molecule that can be transformed or introduced into a mammalian cell such as a T cell and be transcribed and translated to produce a product (e.g., a chimeric receptor).

A. Chimeric Antigen Receptors

In some embodiments of the invention, a chimeric antigen receptors (CAR) is employed. In specific cases, the CAR comprises a fusion of single-chain variable fragments (scFv) that it is specific for the CD19 antigen, the CD28 costimulatory endodomain and the CD3-zeta endodomain. The exodomain of the CAR may be considered an antigen recognition region and can be anything that binds a given target antigen with high affinity. The CAR may be of any kind, but in specific embodiments the CAR targets CD19. However, the CARs may target any type of tumor-associated antigen expressed on the cell surface of a tumor cell, but in specific embodiments they target B-cell-derived malignancies, such as lymphoma and leukemia. Lung cancer, liver cancer, prostate cancer, pancreatic cancer, colon cancer, skin cancer, ovarian cancer, breast cancer, brain cancer, stomach cancer, kidney cancer, spleen cancer, thyroid cancer, cervical cancer, testicular cancer, and/or esophageal cancer may be targeted using specific CAR molecules.

Although in particular embodiments any suitable endodomain is employed in the chimeric receptors of the invention, in specific embodiments it comprises part or all of the CD28 and zeta chain of CD3 endodomains. In specific embodiments, intracellular receptor signaling domains are those of the T cell antigen receptor complex, such as the zeta chain of CD3, also Fcγ RIII costimulatory signaling domains, CD28, DAP10, CD2, alone or in a series with CD3zeta, for example. In specific embodiments, the intracellular domain (which may be referred to as the cytoplasmic domain) comprises part or all of one or more of TCR Zeta chain, CD28, OX40/CD134, 4-1BB/CD137, FcεRIγ, ICOS/CD278, ILRB/CD122, IL-2RG/CD132, and CD40. One or multiple cytoplasmic domains may be employed, as so-called third generation CARs have at least 2 or 3 signaling domains fused together for additive or synergistic effect, for example.

B. Cytokines

In embodiments of the invention, one or more cytokines are utilized in polynucleotides and cells of the invention. The cytokine is useful at least to overcome the limited capacity of cytotoxic T lymphocytes (including adoptively transferred tumor-specific cytotoxic T lymphocytes) to expand within a tumor microenvironment. Although one could utilize IL-2, it can have systemic toxicity and facilitate expansion of undesired cells. In specific embodiments, cytokines such as IL-15 is employed. This allows local delivery of the cytokine at the tumor site avoiding the toxic effects of systemic administration. Other relevant cytokines in the field of immunotherapy can also be included, such as IL-2, IL-7, IL-12 or IL-21, for example.

C. Inducible Suicide Genes

In some embodiments of the invention, a polynucleotide or cell harboring the polynucleotide utilizes a suicide gene, including an inducible suicide gene to reduce the risk of direct toxicity and/or uncontrolled proliferation. In specific aspects, the suicide gene is not immunogenic to the host harboring the polynucleotide or cell. Although thymidine kinase (TK) may be employed, it can be immunogenic. A certain example of a suicide gene that may be used is caspase-9 or caspase-8 or cytosine deaminase. Caspase-9 can be activated using a specific chemical inducer of dimerization (CID).

D. Detectable Markers

In certain embodiments of the invention a polynucleotide or cell harboring the polynucleotide utilizes a detectable marker so that the cell that harbors the polynucleotide is identifiable, for example for qualitative and/or quantitative purposes. The detectable marker may be detectable by any suitable means in the art, including by flow cytometry, fluorescence, spectophotometry, and so forth. An example of a detectable marker is one that encodes a nonfunctional gene produce but that is still detectable by flow cytometry means, for example, or can be used to select transgenic cells by flow cytometry or magnetic selection.

### II. General Embodiments of the Invention

A polynucleotide according to the present invention can be produced by any means known in the art, though preferably it is produced using recombinant DNA techniques. A nucleic acid sequence encoding the several regions of the chimeric receptor can prepared and assembled into a complete coding sequence by standard techniques of molecular cloning (genomic library screening, PCR, primer-assisted ligation, site-directed mutagenesis, etc.). A nucleic acid sequence encoding the other moieities may be similarly prepared. The resulting nucleic acid is preferably inserted into an expression vector and used to transform a suitable expression host cell line, preferably a T lymphocyte cell line, and most preferably an autologous T lymphocyte cell line, a third party derived T cell line/clone, a transformed humor or xerogenic immunologic effector cell line, for expression of the immunoreceptor. NK cells and LAK cells, LIK cells and stem cells that differentiate into these cells, can also be used.

In a nucleic acid construct employed in the present invention, a promoter, such as the LTR promoter of the retroviral vector, is operably linked to a nucleic acid sequence encoding the particular moieties of the vector, including the chimeric antigen receptor of the present invention, the cytokine and the suicide gene, i.e., they are positioned so as to promote transcription of the messenger RNA from the DNA encoding the gene product. The LTR promoter can be substituted by a variety of promoters for use in T cells that are well-known in the art (e.g., the CD4 promoter disclosed by Marodon, et al. (2003) Blood 101(9):3416-23). The promoter can be constitutive or inducible, where induction is associated with the specific cell type or a specific level of maturation, for example. Alternatively, a number of well-known viral promoters are also suitable. Promoters of interest include the β-actin promoter, SV40 early and late promoters, immunoglobulin promoter, human cytomegalovirus promoter, and the Friend spleen focus-forming virus promoter. The promoters may or may not be associated with enhancers, wherein the enhancers may be naturally associated with the particular promoter or associated with a different promoter.

The sequence of the open reading frame encoding the gene products can be obtained from a genomic DNA source, a cDNA source, or can be synthesized (e.g., via PCR), or combinations thereof. Depending upon the size of the genomic DNA and the number of introns, it may be desirable to use cDNA or a combination thereof as it is found that introns stabilize the mRNA or provide T cell-specific expression (Barthel and Goldfeld (2003) J. Immunol. 171(7):3612-9). Also, it may be further advantageous to use endogenous or exogenous non-coding regions to stabilize the mRNA. Sequences of particular mammalian genes are easily obtainable from the National Center for Biotechnology Information database GenBank®.

For expression of a chimeric receptor of the present invention, for example, the naturally occurring or endogenous transcriptional initiation region of the nucleic acid sequence encoding N-terminal component of the chimeric receptor can be used to generate the chimeric receptor in the target host. Alternatively, an exogenous transcriptional initiation region can be used that allows for constitutive or inducible expression, wherein expression can be controlled depending upon the target host, the level of expression desired, the nature of the target host, and the like Likewise, a signal sequence directing the chimeric receptor to the surface membrane can be the endogenous signal sequence of N-terminal component of the chimeric receptor. Optionally, in some instances, it may be desirable to exchange this sequence for a different signal sequence. However, the signal sequence selected should be compatible with the secretory pathway of T cells so that the chimeric receptor is presented on the surface of the T cell. The simultaneous expression of multiple genes in one single vectors may be obtained by the use of 2A sequence peptides derived from foot-and-mouth disease virus to allow transcription and expression of one single mRNA molecule. The sequences of the 2A-like peptides were: pSTA1-TaV RAEGRGSLLTCGDVEENPGP and pSTA1-ERAV QCTNYALLKLAGDVESNPGP^{22,23} (Donnelly M L et al. J Gen Virol. 2001; 82:1027-1041; Szymczak A L et al. Nat Biotechnol. 2004; 22:589-594). The termination region of the entire cassette may be provided by the naturally occurring or endogenous transcriptional termination region of the nucleic acid sequence encoding the C-terminal component of the last gene. Alternatively, the termination region may be derived from a different source. For the most part, the source of the termination region is generally not considered to be critical to the expression of a recombinant protein and a wide variety of termination regions can be employed without adversely affecting expression.

As will be appreciated by one of skill in the art, in some instances one or more of the moieities may be manipulated, such as to increase or decrease amino acids or alter them. The deletion or insertion of amino acids may be as a result of the needs of the construction, providing for convenient restriction sites, ease of manipulation, improvement in levels of expression, or the like. In addition, the substitute of one or more amino acids with a different amino acid can occur for similar reasons, usually not substituting more than about five amino acids in any one domain.

The chimeric construct that encodes the different moities according to the invention can be prepared in conventional ways. Because, for the most part, natural sequences may be employed, the natural genes may be isolated and manipulated, as appropriate, so as to allow for the proper joining of the various components. Thus, the nucleic acid sequences encoding for the N-terminal and C-terminal proteins of the chimeric receptor (for example) can be isolated by employing the polymerase chain reaction (PCR), using appropriate primers that result in deletion of the undesired portions of the gene. Alternatively, restriction digests of cloned genes can be used to generate the chimeric construct. In either case, the sequences can be selected to provide for restriction sites which are blunt-ended, or have complementary overlaps.

The various manipulations for preparing the construct can be carried out in vitro and in particular embodiments the construct is introduced into vectors for cloning and expression in an appropriate host using standard transformation or transfection methods. Thus, after each manipulation, the resulting construct from joining of the DNA sequences is cloned, the vector isolated, and the sequence screened to ensure that the sequence encodes the desired chimeric receptor. The sequence can be screened by restriction analysis, sequencing, or the like.

The constructs of the present invention find application in subjects having or suspected of having cancer by reducing the size of a tumor or preventing the growth or re-growth of a tumor in these subjects. Accordingly, the present invention further relates to a method for reducing growth or preventing tumor formation in a subject by introducing a construct of the present invention into an isolated T cell of the subject and reintroducing into the subject the transformed T cell, thereby effecting anti-tumor responses to reduce or eliminate tumors in the subject (although in alternative embodiments allogeneic cells are used). Suitable T cells that can be used include, cytotoxic lymphocytes (CTL), tumor-infiltrating-lymphocytes (TIL) or other cells that are capable of killing target cells when activated. As is well-known to one of skill in the art, various methods are readily available for isolating these cells from a subject. For example, using cell surface marker expression or using commercially available kits (e.g., ISOCELL™ from Pierce, Rockford, Ill.).

It is contemplated that the construct can be introduced into the subject's own T cells as naked DNA or in a suitable vector. Methods of stably transfecting T cells by electroporation using naked DNA are known in the art. See, e.g., U.S. Pat. No. 6,410,319. Naked DNA generally refers to the DNA encoding the genes of the present invention contained in a plasmid expression vector in proper orientation for expression.

Alternatively, a viral vector (e.g., a retroviral vector, adenoviral vector, adeno-associated viral vector, or lentiviral vector) can be used to introduce the genes of the present invention into T cells. Suitable vectors for use in accordance with the method of the present invention are non-replicating in the subject's T cells. A large number of vectors are known that are based on viruses, where the copy number of the virus maintained in the cell is low enough to maintain the viability of the cell. Illustrative vectors include the pFB-neo vectors (STRATAGENE®) disclosed herein as well as vectors based on HIV, SV40, EBV, HSV or BPV.

Once it is established that the transfected or transduced T cell is capable of expressing the desired gene product with the desired regulation and at a desired level, it can be determined whether the one or more moieties are functional in the host cell to provide for the desired signal induction. Subsequently, the transduced T cells are reintroduced or administered to the subject to activate anti-tumor responses in the subject. To facilitate administration, the transduced T cells according to the invention can be made into a pharmaceutical composition or made implant appropriate for administration in vivo, with appropriate carriers or diluents, which further can be pharmaceutically acceptable. The means of making such a composition or an implant have been described in the art (see, for instance, Remington's Pharmaceutical Sciences, 16th Ed., Mack, ed. (1980)). Where appropriate, the transduced T cells can be formulated into a preparation in semisolid or liquid form, such as a capsule, solution, injection, inhalant, or aerosol, in the usual ways for their respective route of administration. Means known in the art can be utilized to prevent or minimize release and absorption of the composition until it reaches the target tissue or organ, or to ensure timed-release of the composition. Desirably, however, a pharmaceutically acceptable form is employed which does not ineffectuate the cells of the invention. Thus, desirably the transduced T cells can be made into a pharmaceutical composition containing a balanced salt solution, preferably Hanks' balanced salt solution, or normal saline.

A pharmaceutical composition of the present invention can be used alone or in combination with other well-established agents useful for treating cancer. Whether delivered alone or in combination with other agents, the pharmaceutical composition of the present invention can be delivered via various routes and to various sites in a mammalian, particularly human, body to achieve a particular effect. One skilled in the art will recognize that, although more than one route can be used for administration, a particular route can provide a more immediate and more effective reaction than another route. For example, intradermal delivery may be advantageously used over inhalation for the treatment of melanoma. Local or systemic delivery can be accomplished by administration comprising application or instillation of the formulation into body cavities, inhalation or insufflation of an aerosol, or by parenteral introduction, comprising intramuscular, intravenous, intraportal, intrahepatic, peritoneal, subcutaneous, or intradermal administration.

A composition of the present invention can be provided in unit dosage form wherein each dosage unit, e.g., an injection, contains a predetermined amount of the composition, alone or in appropriate combination with other active agents. The term unit dosage form as used herein refers to physically discrete units suitable as unitary dosages for human and animal subjects, each unit containing a predetermined quantity of the composition of the present invention, alone or in combination with other active agents, calculated in an amount sufficient to produce the desired effect, in association with a pharmaceutically acceptable diluent, carrier, or vehicle, where appropriate. The specifications for the novel unit dosage forms of the present invention depend on the particular pharmacodynamics associated with the pharmaceutical composition in the particular subject.

Desirably an effective amount or sufficient number of the isolated transduced T cells is present in the composition and introduced into the subject such that long-term, specific, anti-tumor responses are established to reduce the size of a tumor or eliminate tumor growth or regrowth than would otherwise result in the absence of such treatment. Desirably, the amount of transduced T cells reintroduced into the subject causes a 10%, 20%, 30%, 40%, 50%, 60%, 70%, 80%, 90%, 95%, 98%, or 100% decrease in tumor size when compared to otherwise same conditions wherein the transduced T cells are not present.

Accordingly, the amount of transduced T cells administered should take into account the route of administration and should be such that a sufficient number of the transduced T cells will be introduced so as to achieve the desired therapeutic response. Furthermore, the amounts of each active agent included in the compositions described herein (e.g., the amount per each cell to be contacted or the amount per certain body weight) can vary in different applications. In general, the concentration of transduced T cells desirably should be sufficient to provide in the subject being treated at least from about 1×10⁶ to about 1×10⁹ transduced T cells, even more desirably, from about 1×10⁷ to about 5×10⁸ transduced T cells, although any suitable amount can be utilized either above, e.g., greater than 5×10⁸ cells, or below, e.g., less than 1×10⁷ cells. The dosing schedule can be based on well-established cell-based therapies (see, e.g., Topalian and Rosenberg (1987) Acta Haematol. 78 Suppl 1:75-6; U.S. Pat. No. 4,690,915) or an alternate continuous infusion strategy can be employed.

These values provide general guidance of the range of transduced T cells to be utilized by the practitioner upon optimizing the method of the present invention for practice of the invention. The recitation herein of such ranges by no means precludes the use of a higher or lower amount of a component, as might be warranted in a particular application. For example, the actual dose and schedule can vary depending on whether the compositions are administered in combination with other pharmaceutical compositions, or depending on interindividual differences in pharmacokinetics, drug disposition, and metabolism. One skilled in the art readily can make any necessary adjustments in accordance with the exigencies of the particular situation.

### III. Pharmaceutical Preparations

Pharmaceutical compositions of the present invention comprise an effective amount of one or more cells of the invention dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one antimicrobial composition will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The cells may be dispersed in different types of carriers depending on its administration route. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In further embodiments, the present invention may concern the use of a pharmaceutical lipid vehicle compositions that include the cells, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the antimicrobial composition may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

### IV. Delivery of Cells

In embodiments of the invention, cells generated by methods of the invention are delivered to a mammal. Cell delivery vehicles are known in the art and may be employed to deliver cells of the invention. T cells, NK or LAK cells modified with the vectors encoding the genes of the present invention are usually infused intravenously or in body cavities site of specific disease and are resuspended in saline solutions before infusion.

Suitable doses for a therapeutic effect may be determined by standard means in the art. In specific embodiments, suitable doses are between about 10⁶ and about 10⁹ cells per dose, as an example, preferably in a series of dosing cycles. A preferred dosing regimen may comprise multiple one-week dosing cycles of escalating doses, starting at about 10⁶ cells on Day 0, increasing incrementally up to a target dose of about 10⁹ cells at a later time point. Suitable modes of administration include intravenous, intracavitary (for example by reservoir-access device), intraperitoneal, and direct injection into a tumor mass.

### V. Kits of the Invention

Any of the compositions described herein may be comprised in a kit. The kits will thus comprise, in suitable container means, cells or vectors or related reagents of the present invention. In some embodiments, the kit further comprises an additional agent for treating cancer, and the additional agent may be combined with the vector(s) or cells of the invention or may be provided separately in the kit. In some embodiments, means of taking a sample from an individual and/or of assaying the sample may be provided in the kit. In certain embodiments the kit comprises cells, buffers, cell media, vectors, primers, restriction enzymes, salts, and so forth, for example.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the antimicrobial composition and any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred. The compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit. However, the components of the kit may be provided as dried powder(s). When reagents and/or components are provided as a dry powder, the powder can be reconstituted by the addition of a suitable solvent. It is envisioned that the solvent may also be provided in another container means.

### EXAMPLES

The following examples are offered by way of example and are not intended to limit the scope of the invention in any manner.

### Example 1

T lymphocytes expressing a chimeric antigen receptor (CAR) targeting the CD19 antigen (CAR.19) is of value for the therapy of B-cell malignancies, in particular embodiments of the invention. Because the in vivo survival, expansion and anti-lymphoma activity of CAR.19⁺ T cells remain suboptimal even when the CAR contains a CD28 costimulatory endodomain, the inventors generated a novel construct that also incorporates the interleukin-15 (IL-15) gene and an inducible caspase-9-based suicide gene (iC9/CAR.19/IL-15). Compared with CAR.19⁺ T cells, iC9/CAR.19/IL-15⁺ T cells had: (1) greater numeric expansion upon antigen stimulation (10-fold greater expansion in vitro, and 3- to 15-fold greater expansion in vivo) and reduced cell death rate (Annexin-V⁺/7-AAD⁺ cells 10±6% for iC9/CAR.19/IL-15⁺ T cells and 32±19% for CAR.19⁺ T cells); (2) reduced expression of the programmed death 1 (PD-1) receptor upon antigen stimulation (PD-1⁺ cells o15% for iC9/CAR.19/IL-15⁺ T cells versus 440% for CAR.19⁺ T cells); and (3) improved antitumor effects in vivo (from 4.7- to 5.4-fold reduced tumor growth). In addition, iC9/CAR.19/IL-15⁺ T cells were efficiently eliminated upon pharmacologic activation of the suicide gene. In summary, this strategy safely increases the anti-lymphoma/leukemia effects of CAR.19-redirected T lymphocytes and is a useful approach for treatment of patients with B-cell malignancies.

### Example 2

### Exemplary Materials and Methods

Cell Lines

The following cell lines were used: Daudi and Raji (CD19⁺ Burkitt lymphoma cell lines), HDLM-2 (CD30⁺CD19− Hodgkin's lymphoma cell line), Karpas-299 (CD30⁺CD19− anaplastic lymphoma cell line) and K562 (chronic erythroid leukemia cell line). All cells were purchased from American Type Culture Collection and maintained in culture in RPMI-1640 (Gibco-BRL, San Francisco, Calif., USA) supplemented with 10% fetal bovine serum (Hyclone, Waltham, Mass., USA) and 2 mM L-glutamine (Gibco-BRL).

Plasmid Construction and Retrovirus Production

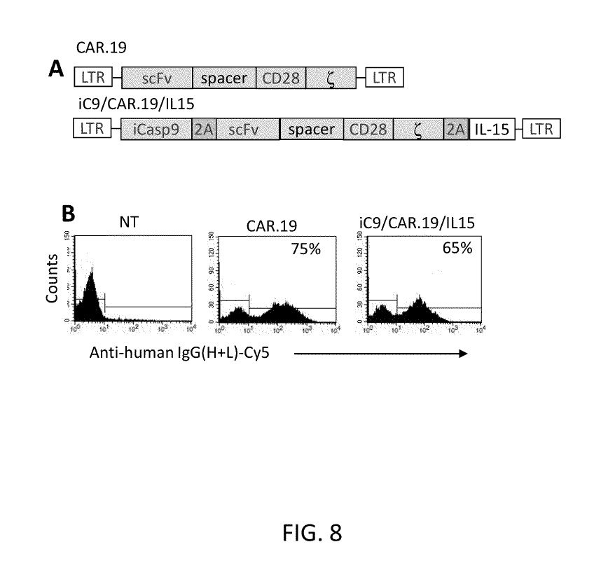

The cassette encoding the single-chain antibody targeting CD19,²⁸ the CD28 endodomain¹⁰ and the ζ-chain of the T-cell receptor complex¹⁰ was cloned into the SFG retroviral backbone to generate the CAR.19 retroviral vector (FIG. 8A). We then generated a second retroviral vector encoding the same CD19-specific CAR in combination with the human IL-15 gene²⁷ and the inducible caspase-9 suicide gene that induces apoptosis upon specific binding with the small molecule dimerizer Chemical inducer of dimerization (CID) AP20187.²⁶ The three genes were linked together using 2A sequence peptides derived from foot-and-mouth disease virus,²⁷ and cloned into the SFG retroviral vector to generate the CAR coexpressed with IL-15 and the inducible suicide gene caspase-9 (iC9/CAR.19/IL-15) retroviral vector (FIG. 8). The vectors encoding FireFly luciferase (FFLuc) and the fusion protein, enhanced green florescent protein-FFLuc (eGFP-FFLuc), used for in vivo imaging have been described previously.^{4,10} Transient retroviral supernatants were produced as previously described.¹⁰

Generation of CAR-Modified T Cells

Peripheral blood mononuclear cells were obtained from four healthy donors and three patients with chronic lymphocytic leukemia (B-CLL) according to the approved protocols of the local institutional review board. Peripheral blood mononuclear cells or CD3⁺-enriched T cells (Miltenyi, Bergisch Gladbach, Germany) for samples collected from B-CLL patients 10 were activated with OKT3 (Ortho Biotech, Bridgewater, N.J., USA) and CD28 (Becton Dickinson, Mountain View, Calif., USA) antibodies and recombinant human IL-2 (100 U/ml) (Proleukin; Chiron, Emeryville, Calif., USA) in complete media (RPMI-1640 (Gibco-BRL) 45%, Click medium (Irvine Scientific, Santa Ana, Calif., USA) 45%, supplemented with 10% fetal calf serum (Hyclone) and 2 mM L-glutamine (GIBCO-BRL)).¹⁰ Activated T cells were transduced with retroviral supernatants on day 3 in plates coated with recombinant fibronectin fragment (FN CH-296; Retronectin; Takara Shuzo, Otsu, Japan).¹⁰ After transduction, T cells were expanded using IL-2 and then used for the studies described below.

Coculture Experiments

Cytokine production. At 7 days after transduction, control non-transduced (NT), CAR.19⁺ and iC9/CAR.19/IL-15⁺ T cells (1×10⁶ cells per well) were cocultured in 24-well plates with B-CLL cells (enriched-CD19⁺ cells) in an effector and tumor cell ratio (E:T) of 1:1. Culture supernatants were collected after 24, 48 and 72 h of culture to measure the production of IL-2, IL-15 and interferon-y using specific enzyme-linked immunosorbent assays (R&D Systems, Inc., Minneapolis, Minn., USA).

T-cell expansion. To evaluate the T-cell growth, control NT, CAR.19⁺ and iC9/CAR.19/IL-15⁺ T cells were maintained in culture and stimulated once a week with CD19⁺ B-CLL cells (E:T ratio of 1:2) without any addition of exogenous cytokines. Cells were cultured for 5 weeks, and counted by Trypan blue exclusion every week.

T-cell division and death. To measure the cell division of T cells upon antigen stimulation, we labeled control NT, CAR.19⁺ and iC9/CAR.19/IL-15⁺ T cells with carboxyfluorescein diacetate succinimidyl ester (CFSE; eBioscience, Inc., San Diego, Calif., USA).²⁹ We then stimulated the T cells with CD19⁺ B-CLL cells (E:T ratio of 2:1) and measured the CFSE dilution by fluorescence-activated cell sorting (FACS) analysis after 5 days of culture. To measure T-cell death upon antigen stimulation, we used the Annexin-V/7-amino-actinomycin (7-AAD) staining and FACS analysis.²⁷

Antitumor effects. To evaluate elimination of tumor cells, control NT, CAR.19⁺ and iC9/CAR.19/IL-15⁺ T cells were cocultured with Daudi cells (CD19⁺) (E:T ratio of 5:1). After 3 days of culture, cells were collected and residual tumor cells were enumerated by FACS analysis. For samples obtained from B-CLL patients, antitumor effects were evaluated against autologous B-CLL cells (E:T ratio of 2:1). In these experiments, B-CLL cells were labeled with CFSE and enumerated by FACS after 3 to 4 days of coculture.¹⁰ In some experiments, we used wild-type Karpas cells (CD30⁺CD19⁻) or CD19⁺ transgenic Karpas as the targets.

Activation of the suicide gene. CID AP20187 (ARIAD Pharmaceuticals, Cambridge, Mass., USA) was kindly provided by Dr Spencer (Baylor College of Medicine) and added at the indicated concentrations to T-cell cultures. The elimination of transgenic cells coexpressing the inducible suicide gene was evaluated 24-48 h after incubation using Annexin-V/7-AAD staining and FACS analysis.²⁷

Immunophenotyping

Cells were stained with fluorescein isothiocyanate-, phycoerythrin- or peridinin-chlorophyll-protein-conjugated monoclonal antibodies. To stain the tumor cells, we used CD19, CD20 and CD30 from Becton Dickinson (Mountain View, Calif., USA). For the T lymphocytes, we used CD3, CD4, CD8, CD56, CD45RA, CD45R0, CD62L, CD27, CD28, CCR7, Bcl-2 and programmed death 1 (PD-1) from Becton Dickinson. To detect the expression of CAR.19, we used a monoclonal antibody Fc-specific cyanine-Cy5-conjugated provided by Jackson ImmunoResearch (West Grove, Pa., USA), which recognized the IgG1-CH2CH3 component of the artificial receptor.¹⁰ Apoptosis was measured using Annexin-V and 7-AAD staining (Becton Dickinson). Cells were analyzed by FACScan (Becton Dickinson), equipped with the filter set for triple fluorescence signals.

Chromium Release Assay

We used 4-h ⁵¹Cr-release assays to evaluate the cytotoxic activity of control and CAR⁺ T lymphocytes.¹⁰ The labeled targets tested included Daudi (CD19⁺ target), HDLM-2 (CD19− target) and K562 (natural killer cell target).

Xenogeneic Lymphoma Models

To assess the persistence and antitumor effect of CAR⁺ T cells in vivo, we used a severe combined immunodeficient (SCID)-lymphoma human xenograft model. Mouse experiments were performed in accordance with the Baylor College of Medicine animal husbandry guidelines according to the approved protocol of the institutional animal care and use committee.

Trafficking and expansion of CAR⁺ cells. In the first set of experiments to evaluate engraftment, Daudi and Raji cells were labeled with FFLuc. SCID mice (8-10-week old; Harlan Sprague Dawley Inc., Indianapolis, Ind., USA) were sublethally irradiated (250 rad) and injected intravenously (i.v.) with either Daudi (3×10⁶) or Raji (2×10⁵) cells. Tumor engraftment was measured using the in vivo imaging system as previously described.^{4,10,27} In brief, mice were injected intraperitoneally (i.p.) with D-luciferin (150 mg/kg), and analyzed using the Xenogen-IVIS Imaging System (Caliper Life Sciences, Hopkinton, Mass., USA). Signal intensity was measured as total photon/sec/cm²/sr (p/s/cm²/sr) as previously described.^{27,29,30} In the second set of experiments to evaluate the in vivo trafficking and persistence of CAR⁺ cells, either control or CAR.19⁺ or iC9/CAR.19/IL-15⁺ cells were labeled with eGFP-FFLuc gene.^{4,27} At 7 days after engraftment with unlabeled Daudi or Raji cells, mice received i.v. 10×10⁶ T cells. No exogenous cytokines were administered to the mice. Trafficking, persistence and expansion of labeled T cells were measured using the Xenogen-IVIS Imaging System.^{4,10,27}

Antitumor effect of CAR⁺ T cells. To measure the antitumor effects of CAR⁺ T cells, mice were engrafted either i.p. or subcutaneously with Daudi cells (1×10⁶ cells) labeled with the FFLuc gene. After 10 days, when the tumor was consistently measurable by light emission, mice received either control NT or CAR.19⁺ or iC9/CAR.19/IL-15⁺ T cells (10×10⁶; 2 doses, 1 week apart). For these experiments we used unlabeled T cells. We evaluated tumor growth using the Xenogen-IVIS Imaging System.^{4,10,27}

In vivo validation of the suicide gene. To evaluate the functionality of the suicide gene, mice bearing tumor cells and receiving iC9/CAR.19/IL-15⁺ T cells labeled with the eGFP-FFluc gene were treated with CID (50 mg) i.p. 2 to 3 doses every other day.²⁷ CID treatment was initiated when the T-cell bioluminescent signal was exponentially increasing, indicating active expansion of the transgenic cells. Mice were then imaged as described above.

Statistical Analysis

Student's t-test was used to determine the statistical significance of differences between samples, and P<0.05 was accepted as indicating a significant difference. For the bioluminescence experiments, intensity signals were summarized using mean±s.d. at baseline and multiple subsequent time points for each group of mice. Changes in intensity of signal from baseline at each time point were calculated and compared using the Wilcoxon signed-ranks test.^{27,29}

### Example 3

### T Lymphocytes Transduced with IC9/CAR.19/IL-15 Vector Release IL-15 After Antigen Stimulation and have Greater Expansion than T Cells Transduced with the CAR.19 Vector

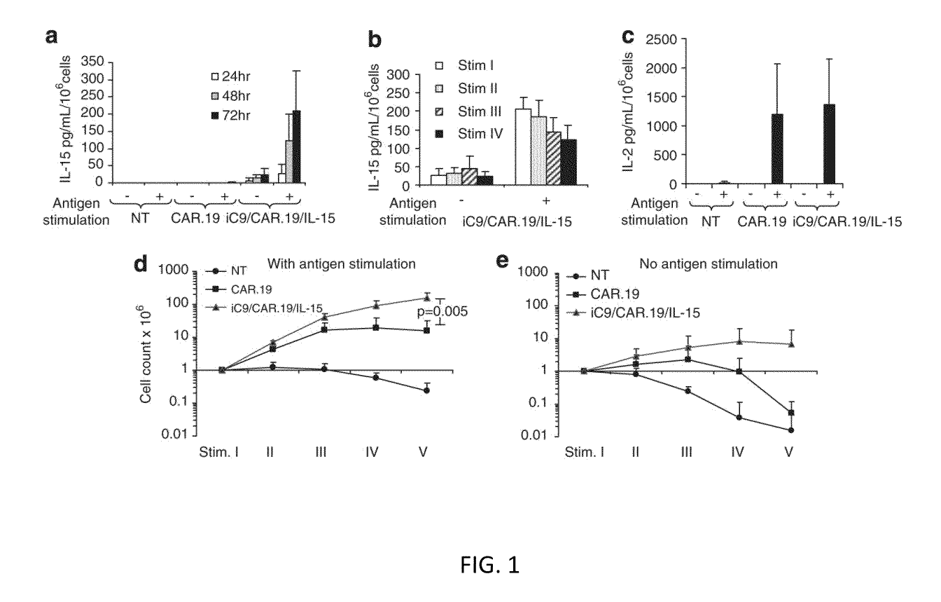

Activated T lymphocytes were equally transduced with one of two retroviral vectors encoding either CAR.19 or iC9/CAR.19/IL-15 (FIG. 8). We then measured IL-15 production by iC9/CAR.19/IL-15⁺ T cells and determined whether production was antigen dependent. Control NT, CAR.19⁺ and iC9/CAR.19/IL-15⁺ T lymphocytes were cultured with or without CD19⁺ target cells (B-CLL).¹⁰ Culture supernatants were collected at multiple time points to measure IL-15 release. As shown in FIG. 1a, IL-15 was undetectable in supernatants collected from stimulated or unstimulated control NT and CAR.19⁺ T cells. In contrast, iC9/CAR.19/IL-15⁺ T cells produced small amounts of IL-15 in the absence of antigen stimulation (25 pg/ml/10⁶ cells (range 3-47 pg/ml)), which significantly increased 72 h after antigen stimulation (240 pg/ml/10⁶ cells (range 110-380 pg/ml); P<0.001). Importantly, when iC9/CAR.19/IL-15⁺ T cells were maintained in culture for more than 4 weeks by weekly stimulation with CD19⁺ B-CLL cells, we found that the production of IL-15 was sustained upon each antigen stimulation (FIG. 1b). As CAR.19 contains the costimulatory endodomain CD28, we also measured IL-2 production by genetically modified T cells.^{10,14,15} As shown in FIG. 1c, incorporation of IL-15 within the iC9/CAR.19/IL-15 vector did not compromise the production of IL-2 by iC9/CAR.19/IL-15⁺ T cells after antigen stimulation (mean 1527 pg/ml/10⁶ cells, range 740-2000 for CAR.CD19⁺ cells and mean 1643 pg/ml/10⁶ cells, range 631-2000 for iC9/CAR.19/IL-15⁺ T cells; P=0.8).

To evaluate whether IL-15 and IL-2 production by iC9/CAR.19/IL-15⁺ T cells increased their expansion compared with CAR.19⁺ cells (which produce only IL-2 in response to antigen stimulation; FIGS. 1a and c), we maintained both CAR.19⁺ and iC9/CAR.19/IL-15⁺ T cells in culture by stimulating them weekly with CD19⁺ B-CLL cells. As shown in FIG. 1d, iC9/CAR.19/IL-15⁺ T cell numbers increased 10-fold compared with CAR.19⁺ T cells (157×10⁶±66×10⁶ total cells vs 15×10⁶±16×10⁶ total cells, respectively; P=0.005) after 5 weeks of culture. In contrast, neither CAR.CD19⁺ T cells nor iC9/CAR.19/IL-15⁺ T cells significantly expanded in the absence of antigen stimulation (FIG. 1e). The viability of iC9/CAR.19/IL-15⁺ T cells in the absence of antigen stimulation was, however, preserved for long term (4-5 weeks) compared with control NT or CAR.19⁺ T cells (FIG. 1e). Expanded CAR.19⁺ and iC9/CAR.19/IL-15⁺ T cells contained both naive (CD45RA⁺) and memory (CD45RO⁺) CD4⁺ and CD8⁺ T lymphocytes, with circa 20% of the latter retaining CD62L and CCR7 expression (data not shown).

### Example 4

### Transgenic Expression of IL-15 Enhances the Survival of CAR-Modified T Cells

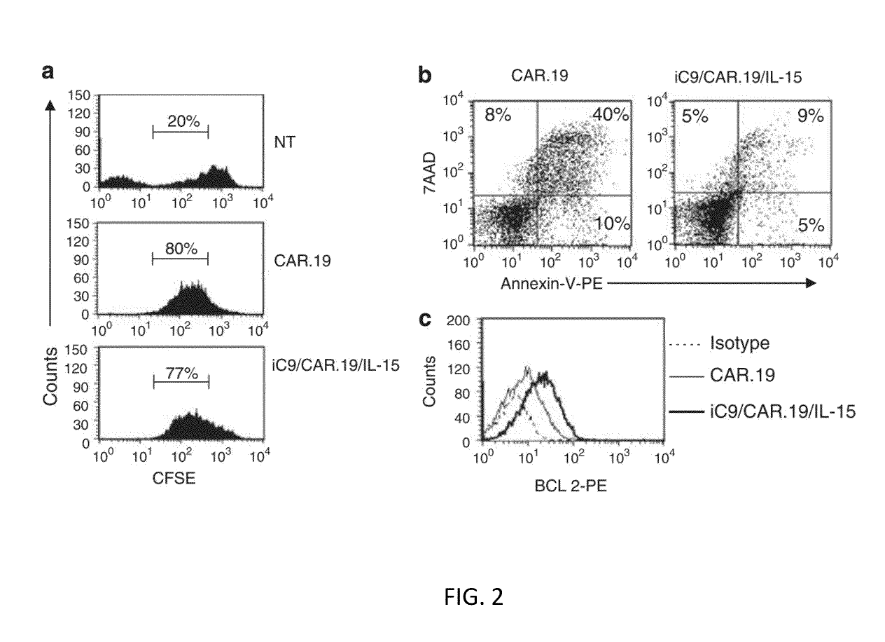

To distinguish whether the greater number of iC9/CAR.19/IL-15⁺ T cells compared with CAR.19⁺ T cells after antigen stimulation was due to increased proliferation or reduced cell death, we analyzed the proliferation and apoptosis of T cells upon antigen stimulation, using CFSE- and Annexin-V/7-AADbased assays, respectively. To measure cell division, T cells were labeled with CFSE and then stimulated with CD19⁺ B-CLL cells. As shown in FIG. 2a, after 5 days of culture, CFSE dilution was comparable for CAR.CD19⁺ and iC9/CAR.19/IL-15⁺ T cells (77±20 and 65±20%, respectively, P=0.07), suggesting similar rates of cell division. In contrast, the death rate of iC9/CAR.19/IL-15⁺ T cells was reduced 5 days after antigenic stimulation, as assessed by Annexin-V/7-AAD staining (annexin-r/7-AAD⁺ cells were 32±19 and 10±6% for CAR.19⁺ and iC9/CAR.19/IL-15⁺ T cells, respectively, Po0.001; FIG. 2b). The improved viability of transgenic cells producing IL-15 also correlated with an increased expression of antiapoptotic genes, such as Bcl-2 (FIG. 2c).

### Example 5

### IC9/CAR.19/IL-15⁺ T Lymphocytes have Enhanced Expansion In Vivo

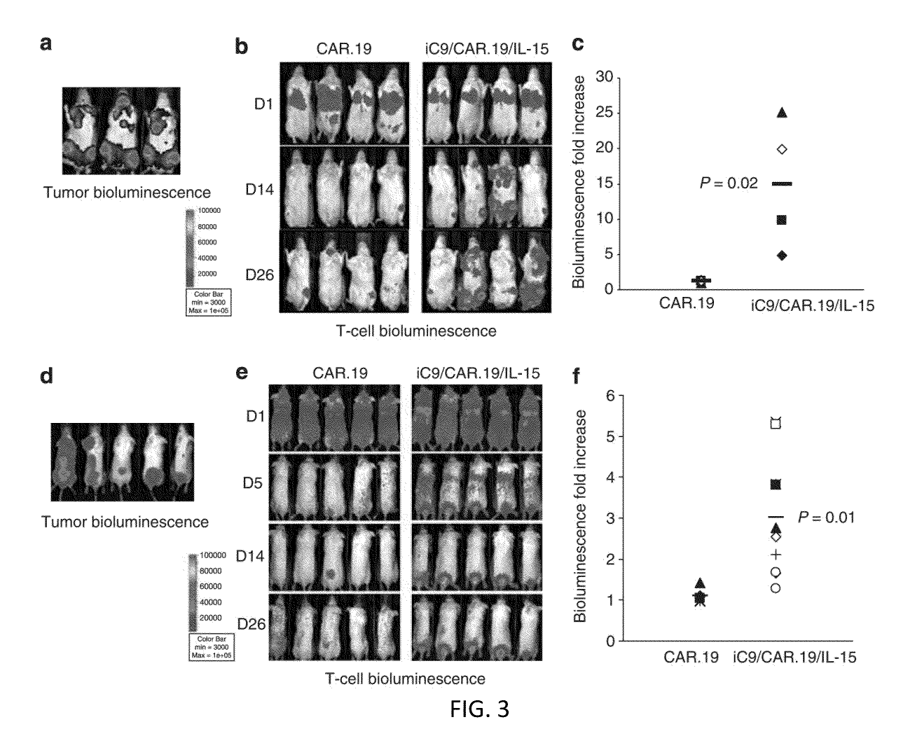

To evaluate the trafficking and persistence of our modified T cells in vivo, we used a SCID mouse lymphoma xenograft, and an extensively validated bioluminescence imaging system.^{4,27,29} We began by evaluating the tumor engraftment after i.v. inoculation of either Daudi and Raji, both of which are CD19⁺ lymphoma cell lines, labeled with FFLuc. We found that 5 days after infusion, Daudi (3×10⁶ cells) had engrafted diffusely in bone marrow, lymphnodes and spleen (FIG. 3a), whereas Raji (2×10⁵ cells) had preferentially engrafted in the spinal cord (FIG. 3d). Tumor localization was confirmed by phenotypic analysis of biopsy samples (data not shown). After defining the timing and sites of tumor engraftment, we assessed T-cell trafficking to the tumor and T-cell persistence in vivo. Control NT, CAR.19⁺ and iC9/CAR.19/IL-15⁺ T cells were labeled with eGFP-FFLuc,^{10,27} and infused (10×10⁶ cells/mouse) in mice previously engrafted with either unlabeled Daudi or Raji cells. FIGS. 3b and e illustrate that both CAR.19⁺ and iC9/CAR.19/IL-15⁺ T cells localized at the tumor site, as T-cell bioluminescence had superimposable anatomical localizations for the labeled tumor cells (FIGS. 3a and d). T-cell bioluminescence remained barely detectable when Daudi- or Raji-engrafted tumor cells were treated with labeled control NT T cells. Importantly, although the bioluminescence signal corresponding to CAR.19⁺ T cells only modestly increased by day 25 after infusion (from 4×10⁵±8×10³ to 4×10⁵±4×10⁴ for mice engrafted with Daudi, and from 1×10⁵±2×10⁴ to 1.2×10⁵±3×10⁴ for mice engrafted with Raji), corresponding to 1.1- and 1-fold increase, respectively (FIGS. 3c and f), the signal from iC9/CAR.19/IL-15⁺ T cells significantly increased over the following 25 days (from 5×10⁵±8×10⁴ to 9×10⁶±3×10⁶ for mice engrafted with Daudi and from 5×10⁵±8×10⁴ to 2×10⁶±1×10⁶ for mice engrafted with Raji), corresponding to 15- and 3-fold increase, respectively (P=0.02 and P=0.01; FIGS. 3c and f), showing increased expansion and persistence of these cells.

### Example 6

### T Lymphocytes Transduced with the IC9/CAR.19/IL-15 Vector have Enhanced Antitumor Activity

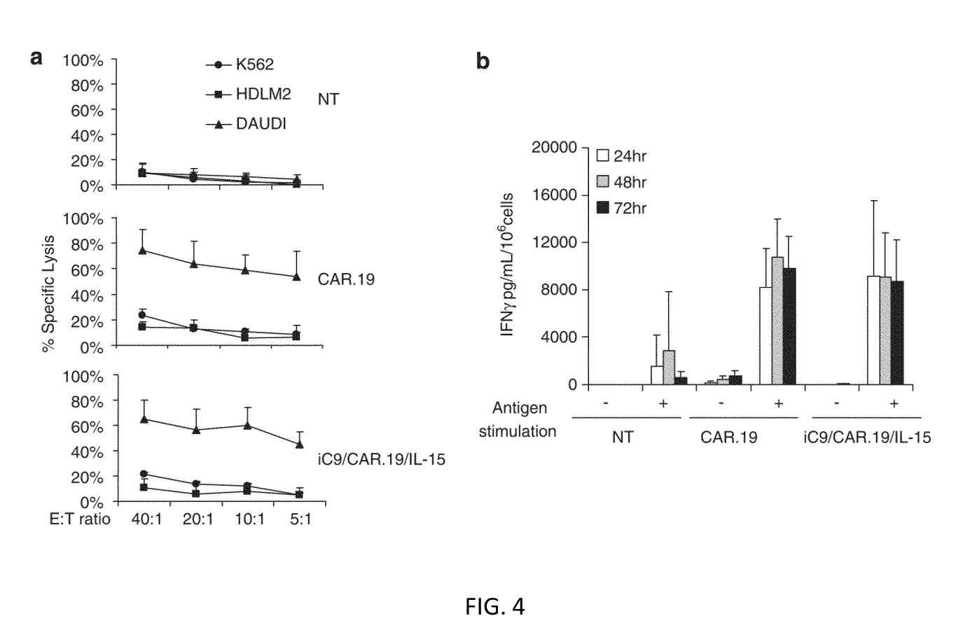

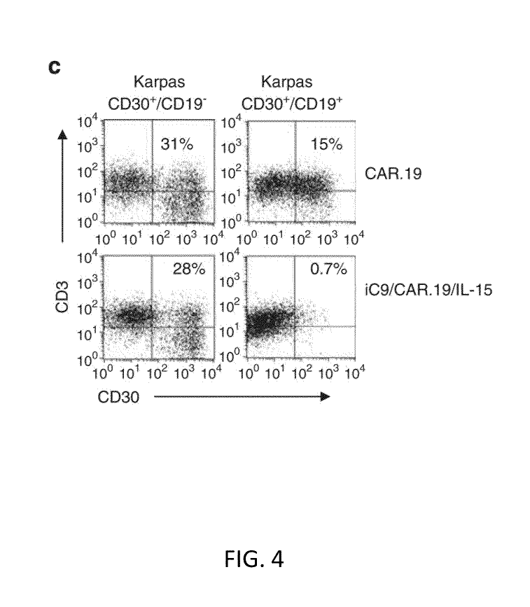

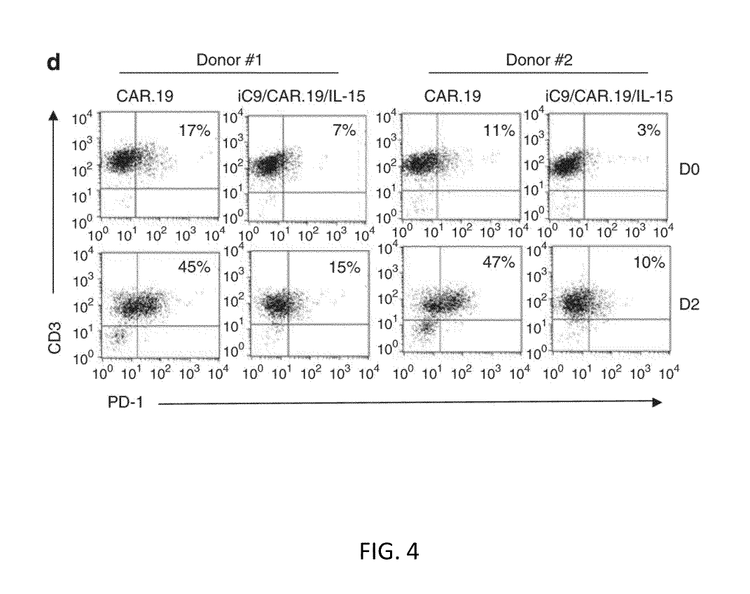

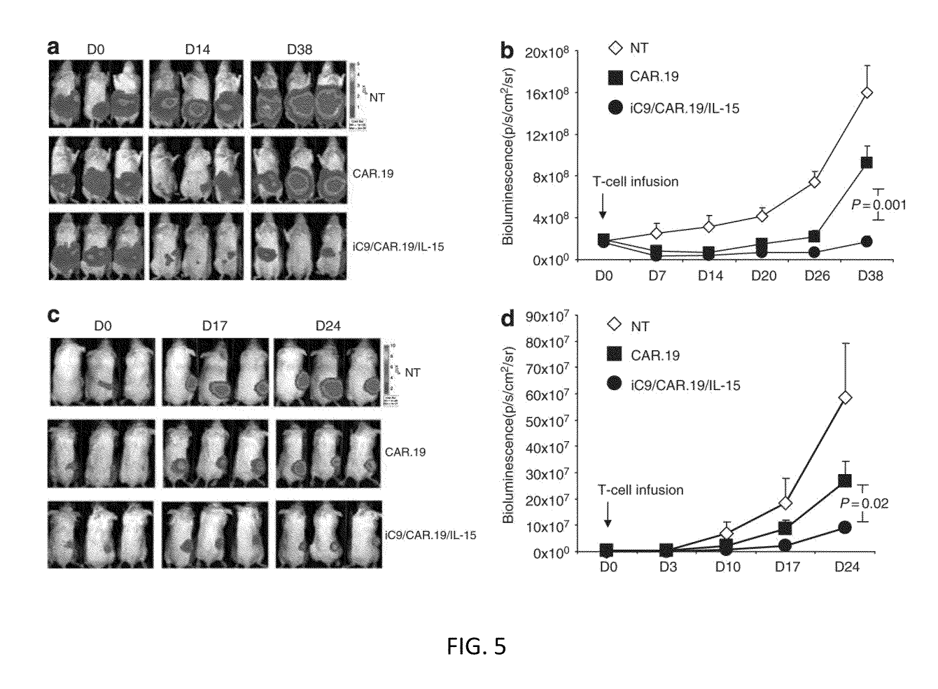

We measured the cytotoxic activity of T cells against CD19⁺ and CD19− tumor cell lines using standard ⁵¹Cr-release assays. Both CAR.CD19⁺ and iC9/CAR.19/IL-15⁺ T cells had equal and specific cytotoxic activity against CD19⁺ Daudi cells (63±17 and 57±16% specific lysis in an E:T ratio of 20:1, respectively), with <13% killing of HDLM-2 (CD19−) and erythroleukemia-derived K562 (natural killer cell target) cell lines (FIG. 4a). Control T cells showed no significant cytotoxicactivity against any of these target cell lines. In parallel, both CAR.19⁺ and iC9/CAR.19/IL-15⁺ T cells produced equal amounts of interferon-γ in response to CD19⁺ tumor cells (mean of 8327 pg/ml/10⁶ cells, range 5080-12 510 for CAR.CD19⁺ cells and of 9147 pg/ml/10⁶ cells, range 3025-18 010 for iC9/CAR.19/IL-15⁺ cells; FIG. 4b). To further confirm that IL-15 production by iC9/CAR.19/IL-15⁺ T cells enhanced the elimination of tumor cells through the CAR, we cocultured T cells with wild-type Karpas-299 tumor cells (CD30⁺CD19⁻), or with Karpas cells modified to stably express the CD19 molecule (Karpas CD30⁺CD19⁺). After 4 to 5 days in an initial T cell and tumor cell ratio of 5:1, residual tumor cells were quantified by FACS analysis enumerating CD30⁺ tumor cells. Both CAR.19⁺ and iC9/CAR.19/IL-15⁺ T cells efficiently eliminated Karpas CD19⁺ tumor cells when T-cell antitumor activity was evaluated using T-cell lines maintained in short-term culture (1 week) after transduction. In contrast, when T-cell lines were maintained in culture for 4 weeks, and stimulated weekly with CD19⁺ B-CLL cells, only iC9/CAR.19/IL-15⁺ T cells maintained their ability to completely eliminate tumor cells from the culture (residual tumor cells 1±0.7 and 10±5% for iC9/CAR.19/IL-15⁺ T cells and CAR.19⁺ T cells, respectively; P=0.001). Importantly, even after 4 weeks of expansion in culture, the antitumor effects of both CAR.19⁺ and iC9/CAR.19/IL-15⁺ T cells remained antigen specific, as they lacked activity against wild-type (CD19⁻) Karpas-299 cells (FIG. 4c). To discover potential mechanisms for the sustained effector function of IL-15-producing cells upon prolonged culture and repeated antigen stimulation, we evaluated the expression of PD-1, a marker of T-cell exhaustion,³¹ and found that iC9/CAR.19/IL-15⁺ T cells had lower expression of PD-1 (PD-1⁺ T cells <15%) 2 days after stimulation with B-CLL cells than CAR.19⁺ T cells (PD-1⁺ T cells 440%; FIG. 4d). The above in vitro data were then corroborated with experiments in vivo. In two different models, SCID mice were engrafted either i.p. or subcutaneously with 3×10⁶ Daudi cells labeled with FFLuc. After 7 days, these mice were treated with two weekly infusions i.p. (for mice engrafted with i.p. tumor) or i.v (for mice engrafted with subcutaneous tumor) of control NT, CAR.19⁺ or iC9/CAR.19/IL-15⁺ T cells (10×10⁶). Tumor growth was monitored by measuring changes in tumor bioluminescence over time. As shown in FIGS. 5a and b, the tumor bioluminescence of mice engrafted i.p. with Daudi cells rapidly increased in recipients of control NT T cells (rising from 1.8×10⁸±4×10⁷ to 16×10⁸±2.6×10⁸ by day 38). CAR.19⁺ T cells transiently controlled tumor growth (from 1.9×10⁸±3×10⁷ to 9.3×10⁸±1.6×10⁸ by day 38), whereas iC9/CAR.19/IL-15⁺ T cells significantly controlled tumor expansion so that signal rose from 1.6×10⁸±3×10⁷ to only 1.7×10⁸±5×10⁸ by day 38 (P=0.001 when compared with mice receiving CAR.19⁺ T cells). Similarly, the tumor bioluminescence of mice engrafted subcutaneously with Daudi cells rapidly increased in mice receiving control NT T cells (from 2.6×10⁵±1.1×10⁴ to 57×10⁷±20×10⁷ by day 24), showed transient control in recipients of CAR.19⁺ T cells (from 3.3×10⁵±9.6×10⁴ to 26×10⁷±7.6×10⁷ by day 24) and greatest control in recipients of iC9/CAR.19/IL-15⁺ T cells (tumor growth from 2.8×10⁵±7×10⁴ to 5.5×10⁷±1.5×10⁷ by day 24; P¼0.02 when compared with mice receiving CAR.19⁺ T cells; FIGS. 5c and d).

### Example 7

### IC9/CAR.19/IL-15⁺ T Cells are Eliminated After Activation of the Suicide Gene by Exposure to the Small-Molecule CID

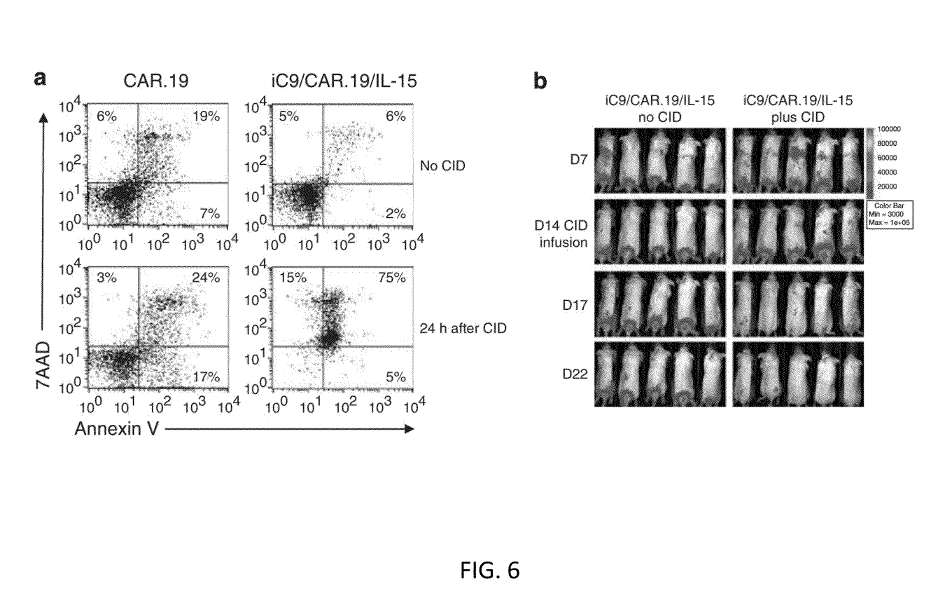

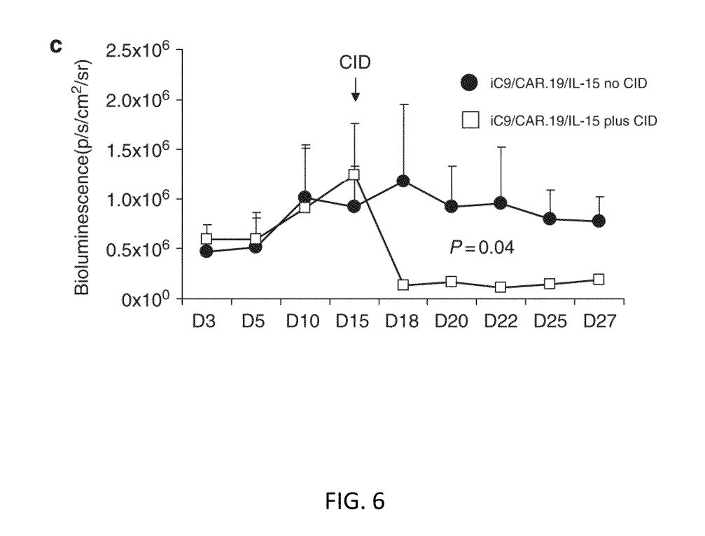

Because the production of an autocrine growth factor raises concerns about autonomous, uncontrolled T-cell growth, we incorporated in our construct a suicide gene based on the inducible caspase-9 gene.^{26,27} As shown in FIG. 6a, the addition of 50 nM CID to cultures of iC9/CAR.19/IL-15⁺ T cells induced apoptosis/necrosis of 495% of transgenic cells within 24 h, as assessed by annexin-V-7AAD staining.²⁷ The suicide gene was also effective in vivo. Mice were engrafted i.v. with Raji tumor cells and then infused with eGFP-FFLuc labeled iC9/CAR.19/IL-15⁺ T cells. These cells localized and expanded at the tumor site by day 14 after infusion as assessed by bioluminescence measurement (FIG. 6b). T-cell bioluminescence drastically reduced (from 1.2×10⁶±7.7×10¹⁵ to 1.3×10⁵±3×10⁴) after administration of the CID, consistent with a significant elimination of the transgenic cells.²⁷

### Example 8

IL-15 Expression Improves Proliferation and Antitumor Effects of CAR.19⁺ T Cells Generated from B-CLL Samples

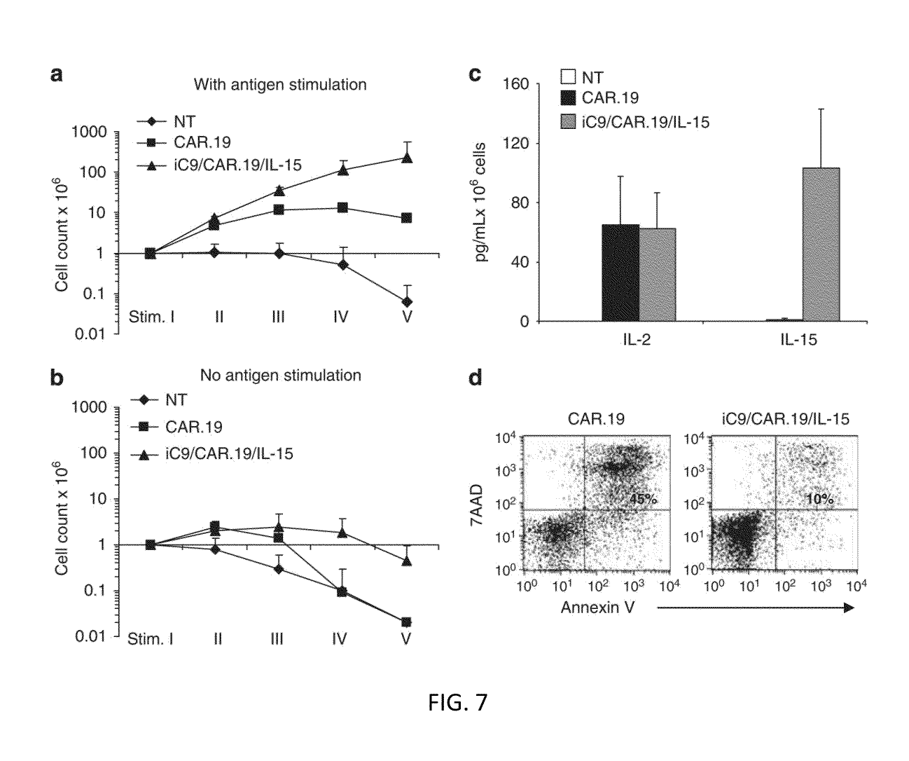

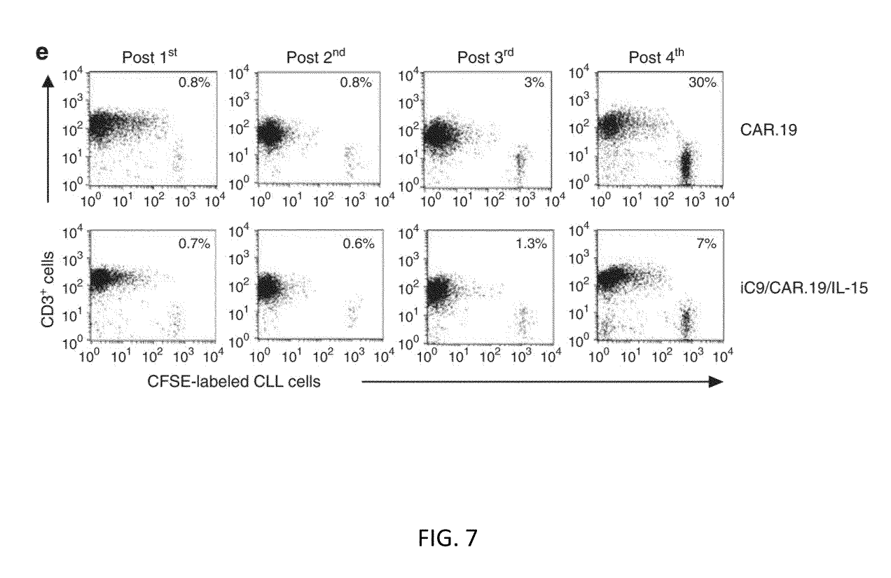

Finally, we validated the efficacy of the combination of CAR.19, IL-15 and the suicide gene in cells obtained from subjects with B-CLL. As shown in FIG. 7a, the expansion of T cells isolated from B-CLL patients was enhanced after transduction with the iC9/CAR.19/IL-15 vector and stimulation with autologous B-CLL cells (E:T ratio of 1:1) compared with CAR.19⁺ T cells (228×10⁶±315×10⁶ total cells vs 7×10⁶±2.7×10⁶ total cells, respectively). Their expansion remained fully antigen dependent as we observed for T-cell lines generated from healthy donors (FIG. 7b). iC9/CAR.19/IL-15⁺ T cells also released both IL-15 (103±39 pg/ml/10⁶ cells) and IL-2 (62±24 pg/ml/10⁶ cells) in response to autologous B-CLL cells, whereas CAR.19⁺ T cells produced only IL-2 (65±33 pg/ml/10⁶ cells; FIG. 7c). Expansion of patients' T cells was mainly driven by the antiapoptotic effect of IL-15 upon antigen stimulation (FIG. 7d). We also confirmed that iC9/CAR.19/IL-15⁺ patient T cells retained enhanced antitumor activity against autologous B-CLL after long-term culture and repeated exposure to autologous tumor cells, whereas CAR.19⁺ patient T cells did not (FIG. 7e).

### Example 9

### Significance of Certain Embodiments of the Invention

We have forced expression of IL-15 in T cells redirected with a CAR that specifically targets the CD19 antigen, and shown that these engineered T cells had superior survival, expansion and antitumor activity in vivo when compared with redirected T cells that only receive CD28 costimulation through the CAR but lack IL-15 production. Importantly, the incorporation of an inducible suicide gene and its pharmacologic activation efficiently eliminated these gene-modified T cells, further increasing the safety of the proposed approach.

In vivo persistence and expansion of adoptively transferred tumor-specific T cells is crucial to obtain sustained clinical responses.³² This is particularly important for T cells engrafted with CARs that lack costimulatory endodomains, as they do not release helper cytokines upon engagement with the antigen expressed by tumor cells.^{10,14,15,33,34} In addition, they cannot receive appropriate activation by professional antigen-presenting cells in secondary lymphoid organs, as the native αβ-cell receptors of these redirected T cells are not generally specific for latent antigens consistently processed and presented by the host antigen-presenting cells.^{4,13} The incorporation of the CD28 costimulatory signaling domain as part of the CAR itseif^{10,14,15,33} can enhance activation and proliferation of these cells secondary to IL-2 secretion, even without cross-presentation by antigen-presenting cells.^{10,15} However, the overall persistence and antitumor effects of such CAR.19-redirectd T cells still remain limited.^{10,14,19} Alternative costimulatory endodomains may be superior to CD28, and incorporation of 4-1BB endodomain, for example,^{16,35} or a combination of both CD28 and 4-1BB, has been reported to increase the persistence and antitumor efficacy over CD28-containing CARs.^{18,21} Nevertheless, we reasoned that the ectopic production of IL-15 by CAR-modified T cells is a valid addition to the incorporation of costimulatory endodomains within the CAR construct as the benefits would occur through different pathways and mechanisms, because neither CD28 nor 4-1BB costimulation lead to the production of IL-15, a cytokine that potently enhances the antitumor activity of effector T cells in vitro and in vivo.^{8,36,37} The approach ensures that CARmodified T cells receive both IL-2 and IL-15 stimulation after chimeric receptor engagement in the tumor microenvironment.

We have accommodated the IL-15 gene in a single retrovirus vector in combination with the CAR.19 encoding the CD28 endodomain and an inducible suicide gene without significantly affecting CAR.19 expression, an essential requirement if tumor specificity is to be maintained. Minimal cytokine is produced when the T cells are unstimulated, but the amount significantly increases after stimulation with tumor cells, and production is maintained by the T cells after more than 4 weeks of culture. This transgenic IL-15 is biologically functional, as iC9/CAR.19/IL-15⁺ T cells have superior expansion after antigen stimulation than control CAR.19⁺ T cells. These benefits are largely attributable to the reduced susceptibility of iC9/CAR.19/IL-15⁺ T cells to cell death induced upon antigen stimulation, likely because these cells have higher expression of the antiapoptotic gene Bcl-238 when compared with control CAR.19⁺ T cells. Importantly, in vitro experiments show that iC9/CAR.19/IL-15⁺ T cells retain enhanced antitumor activity when they are ‘chronically’ exposed to tumor cells. This effect may be determined by an increased protection of iC9/CAR.19/IL-15⁺ T cells from functional exhaustion, as indicated by a lower expression of PD-1 upon antigen stimulation than CAR.19⁺ T cells, as PD-1 has been recognized as a marker of exhausted T cells in chronic infections such as human immunodeficiency virus,³¹ hepatitis C³⁹ and tumorinfiltrating T lymphocytes.^{40,43} The transcriptional or post-transcriptional mechanisms that reduce expression of PD-1 in iC9/CAR.19/IL-15⁺ T cells after repeated antigen stimulation are not known. Nonetheless, the observation in certain embodiments has clinical implications, as CAR-modified T cells that target self-antigens, such as CD19, will inevitably be exposed to a large number of target cells in vivo, and PD-1 ligands may be expressed by either the tumor cell itself^{40,42,43} or by tumor-associated dendritic cells.⁴⁴ The improved antitumor effects observed in vitro were matched by superior in vivo activity, and iC9/CAR.19/IL-15⁺ T cells retained their tumor homing, expanded at tumor sites and had enhanced antitumor activity compared with control CAR.19⁺ T cells.

Recently, intermittent systemic administration of recombinant IL-15 has been shown to induce expansion of memory CD4⁺ and CD8⁺ T cells in nonhuman primates.⁴⁵ Administration of IL-15 is tested in patients to support the expansion of adoptively transferred tumor-specific T cells. The present invention is advantageous as it delivers the cytokine directly to T cells at the tumor site, avoiding the toxicities that may be observed in patients receiving systemic administration of recombinant cytokines, especially when the cytokine is used at high doses.⁴⁶ The invention is also valuable for adoptive transfer of tumor-specific T lymphocytes in lymphodepleted patients⁴⁷ as it ensures long-term availability of IL-15 for tumor-specific T cells to overcome the antiproliferative effect of transforming growth factor-b48 and regulatory T cells⁴⁹ within the tumor microenvironment. Because IL-15 production by gene-modified T cells occurs predominantly after they engage tumor cells through their CARs, the risks of autonomous growth should remain small. Nonetheless, given the concerns regarding retroviral-associated oncogenesis,²⁵ and the potential side effects reported after T-cell therapy with CAR-modified T cells,^{11,50} we incorporated a suicide gene based on the inducible caspase-9 molecule within the construct. Activation of this suicide gene rapidly induces apoptosis of IL-15-producing T cells both in vitro and in vivo. Previous observation that the small fraction of cells (<10%) that seem to escape apoptosis/necrosis shortly after exposure to the dimerizer drug do not express detectable levels of any transgene, including cytokine,²⁷ nor proliferate after antigen stimulation,²⁷ further increases the safety of embodiments of the invention. This suicide gene is already under evaluation in a phase I clinical trial in which patients undergoing haploidentical transplant receive donor-derived T cells gene modified with the inducible caspase-9 gene.⁵¹

In conclusion, transgenic expression of IL-15 improves survival, expansion and antitumor effects of CD19-specific redirected T cells. The incorporation of an effective suicide gene should further assure the safety of the approach and increase its potential clinical applicability.

### Example 10

### Generation of a New “Universal Construct” Encoding IL-15 and the Inducible Caspase9 Suicide Gene

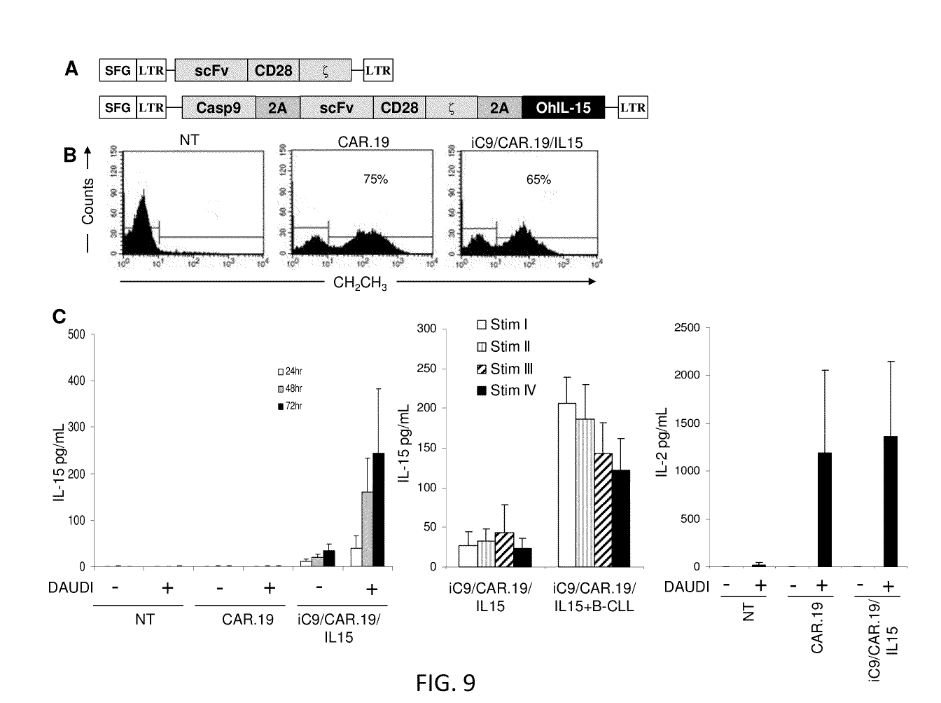

As illustrated in FIG. 9, we previously generated a tricistronic vector in which 3 sequential genes were encoded: icaspase9, CD19-specific CAR and IL-15 (Hoyos et al. (2010; Leukemia 24:1160-1170).

FIG. 9 shows T cells transduced with the iC9/CAR.19/IL-15 vector produce IL-15 in response to antigen stimulation. Panel A illustrates the schemes of the vectors. Panel B illustrates the transduction of T cells. Panel C illustrates the kinetics of IL-15 release by control NT, CAR.19⁺ and iC9/CAR.19/IL-15⁺ T cells with or without antigen stimulation (CD19⁺ B-CLL cells) (left panel). The release of IL-15 by iC9/CAR.19/IL-15⁺ T cells when these cells were maintained in culture for 4 weeks and stimulated once a week with the antigen (CD19⁺ B-CLL cells) (middle panel). The release of IL2 by control NT, CAR.19⁺ and iC9/CAR.19/IL-15⁺ T cells with or without antigen stimulation (CD19⁺ B-CLL cells) (right panel). Data in these panels represent the mean±SD of 4 T-cell lines.

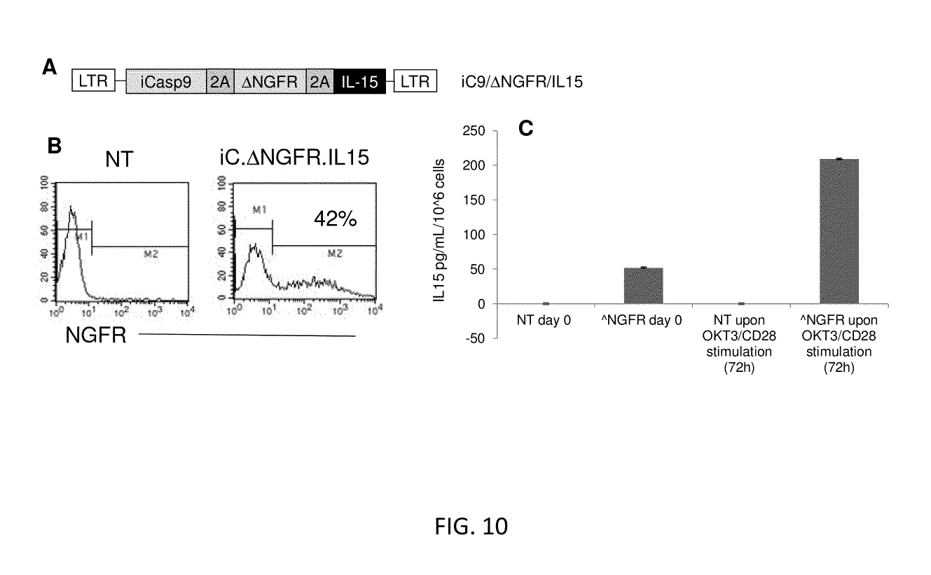

In an effort to generate a vector that can be used for several applications (universal vector) the CAR from the original construct iC9/CAR.19/IL-15 was not employed. Instead, the inventors used a selectable marker based on a truncated form of NGFR to generate the new construct iC9/ΔNGFR/IL-15. The construct is illustrated in FIG. 10. For clinical application this construct can be used as a single vector to transduce antigen-specific cytotoxic T lymphocytes with native αβTCR antigen specificity or it can be used in combination (double transduction) with vectors encoding chimeric antigen receptors (CARs) specific for different antigens. FIG. 10 illustrates that the novel construct allows production of T lymphocytes upon TCR activation.

FIG. 10. T cells transduced with the iC9/ΔNGFR/IL-15 vector produce IL-15. Panel A illustrates the scheme of the new vector. Panel B illustrates the transduction of T cells. Panel C illustrates the IL-15 release by control NT and iC9/ΔNGFR/IL-15⁺ T cells with or without stimulation by immobilized OKT and CD28 antibodies.

### REFERENCES

1 Till B G, Jensen M C, Wang J, Chen E Y, Wood B L, Greisman H A et al. Adoptive immunotherapy for indolent non-Hodgkin lymphoma and mantle cell lymphoma using genetically modified autologous CD20-specific T cells. Blood 2008; 112: 2261-2271.
2 Hombach A, Heuser C, Sircar R, Tillmann T, Diehl V, Pohl C et al. Characterization of a chimeric T-cell receptor with specificity for the Hodgkin's lymphoma-associated CD30 antigen. J Immunother 1999; 22: 473-480.
3 Sadelain M, Brentjens R, Riviere I. The promise and potential pitfalls of chimeric antigen receptors. Curr Opin Immunol 2009; 21: 215-223.
4 Savoldo B, Rooney C M, Di Stasi A, Abken H, Hombach A, Foster A E et al. Epstein Barr virus specific cytotoxic T lymphocytes expressing the anti-CD30{zeta} artificial chimeric T-cell receptor for immunotherapy of Hodgkin disease. Blood 2007; 110: 2620-2630.
5 Dotti G, Savoldo B, Brenner M. Fifteen years of gene therapy based on chimeric antigen receptors: ‘are we nearly there yet?’. Hum Gene Ther 2009; 20: 1229-1239.
6 Eshhar Z, Waks T, Gross G, Schindler D G. Specific activation and targeting of cytotoxic lymphocytes through chimeric single chains consisting of antibody-binding domains and the gamma or zeta subunits of the immunoglobulin and T-cell receptors. Proc Natl Acad Sci USA 1993; 90: 720-724.
7 Cooper L J, Topp M S, Serrano L M, Gonzalez S, Chang W C, Naranjo A et al. T-cell clones can be rendered specific for CD19: toward the selective augmentation of the graft-versus-B-lineage leukemia effect. Blood 2003; 101: 1637-1644.
8 Brentjens R J, Latouche J B, Santos E, Marti F, Gong M C, Lyddane C et al. Eradication of systemic B-cell tumors by genetically targeted human T lymphocytes co-stimulated by CD80 and interleukin-15. Nat Med 2003; 9: 279-286.
9 Jensen M, Tan G, Forman S, Wu A M, Raubitschek A. CD20 is a molecular target for scFvFc:zeta receptor redirected T cells: implications for cellular immunotherapy of CD20⁺ malignancy. Biol Blood Marrow Transplant 1998; 4: 75-83.
10 Vera J, Savoldo B, Vigouroux S, Biagi E, Pule M, Rossig C et al. T lymphocytes redirected against the kappa light chain of human immunoglobulin efficiently kill mature B lymphocyte-derived malignant cells. Blood 2006; 108: 3890-3897.
11 Brentjens R., Treatment of chronic lymphocytic leukemia with genetically targeted autologous T cells: case report of an unforeseen adverse event in a phase I trial. Mole Ther 2010; 18: 666-668.
12 Kershaw M H, Westwood J A, Parker L L, Wang G, Eshhar Z, Mavroukakis S A et al. A phase I study on adoptive immunotherapy using gene-modified T cells for ovarian cancer. Clin Cancer Res 2006; 12 (20 Pt 1): 6106-6115.
13 Pule M A, Savoldo B, Myers G D, Rossig C, Russell H V, Dotti G et al. Virus-specific T cells engineered to coexpress tumor-specific receptors: persistence and antitumor activity in individuals with neuroblastoma. Nat Med 2008; 14: 1264-1270.
14 Kowolik C M, Topp M S, Gonzalez S, Pfeiffer T, Olivares S, Gonzalez N et al. CD28 costimulation provided through a CD19-specific chimeric antigen receptor enhances in vivo persistence and antitumor efficacy of adoptively transferred T cells. Cancer Res 2006; 66: 10995-11004.
15 Maher J, Brentjens R J, Gunset G, Riviere I, Sadelain M. Human T-lymphocyte cytotoxicity and proliferation directed by a single chimeric TCRzeta/CD28 receptor. Nat Biotechnol 2002; 20: 70-75.
16 Imai C, Mihara K, Andreansky M, Nicholson I C, Pui C H, Geiger T L et al. Chimeric receptors with 4-1BB signaling capacity provoke potent cytotoxicity against acute lymphoblastic leukemia. Leukemia 2004; 18: 676-684.
17 Milone M C, Fish J D, Carpenito C, Carroll R G, Binder G K, Teachey D et al. Chimeric receptors containing CD137 signal transduction domains mediate enhanced survival of T cells and increased antileukemic efficacy in vivo. Mol Ther 2009; 17: 1453-1464.
18 Carpenito C, Milone M C, Hassan R, Simonet J C, Lakhal M, Suhoski M M et al. Control of large, established tumor xenografts with genetically retargeted human T cells containing CD28 and CD137 [0001] domains. Proc Natl Acad Sci USA 2009; 106: 3360-3365.
19 Tammana S, Huang X, Wong M, Milone M C, Ma L, Levine B L et al. 4-1BB and CD28 Signaling plays a synergistic role in redirecting umbilical cord blood t cells against B-cell malignancies. Hum Gene Ther 2010; 21: 75-86.
20 Zhao Y, Wang Q J, Yang S, Kochenderfer J N, Zheng Z, Zhong X et al. A herceptin-based chimeric antigen receptor with modified signaling domains leads to enhanced survival of transduced T lymphocytes and antitumor activity. J Immunol 2009; 183: 5563-5574.
21 Wang J, Jensen M, Lin Y, Sui X, Chen E, Lindgren C G et al. Optimizing adoptive polyclonal T cell immunotherapy of lymphomas, using a chimeric T cell receptor possessing CD28 and CD137 costimulatory domains. Hum Gene Ther 2007; 18:712-725.
22 Pule M A, Straathof K C, Dotti G, Heslop H E, Rooney C M, Brenner M K. A chimeric T cell antigen receptor that augments cytokine release and supports clonal expansion of primary human T cells. Mol Ther 2005; 12: 933-941.
23 Ma A, Koka R, Burkett P. Diverse functions of IL-2, IL-15, and IL-7 in lymphoid homeostasis. Annu Rev Immunol 2006; 24: 657-679.
24 Waldmann T A, Dubois S, Tagaya Y. Contrasting roles of IL-2 and IL-15 in the life and death of lymphocytes: implications for immunotherapy. Immunity 2001; 14: 105-110.
25 Hsu C, Jones S A, Cohen C J, Zheng Z, Kerstann K, Zhou J et al. Cytokine-independent growth and clonal expansion of a primary human CD8⁺ T-cell clone following retroviral transduction with the IL-15 gene. Blood 2007; 109: 5168-5177.
26 Straathof K C, Pule M A, Yotnda P, Dotti G, Vanin E F, Brenner M K et al. An inducible caspase 9 safety switch for T-cell therapy. Blood 2005; 105: 4247-4254.
27 Quintarelli C, Vera J F, Savoldo B, Giordano Attianese G M, Pule M, Foster A E et al. Co-expression of cytokine and suicide genes to enhance the activity and safety of tumor-specific cytotoxic T lymphocytes. Blood 2007; 110: 2793-2802.
28 Rossig C, Brenner M K. Chimeric T-cell receptors for the targeting of cancer cells. Acta Haematol 2003; 110: 154-159.
29 Di Stasi A, De Angelis B, Rooney C M, Zhang L, Mahendravada A, Foster A E et al. T lymphocytes coexpressing CCR4 and a chimeric antigen receptor targeting CD30 have improved homing and antitumor activity in a Hodgkin tumor model. Blood 2009; 113: 6392-6402.
30 Kim Y J, Dubey P, Ray P, Gambhir S S, Witte O N. Multimodality imaging of lymphocytic migration using lentiviral-based transduction of a tri-fusion reporter gene. Mol Imaging Biol 2004; 6: 331-340.
31 Day C L, Kaufmann D E, Kiepiela P, Brown J A, Moodley E S, Reddy S et al. PD-1 expression on HIV-specific T cells is associated with T-cell exhaustion and disease progression. Nature 2006; 443: 350-354.
32 Dudley M E, Rosenberg S A. Adoptive-cell-transfer therapy for the treatment of patients with cancer. Nat Rev Cancer 2003; 3: 666-675.
33 Finney H M, Lawson A D, Bebbington C R, Weir A N. Chimeric receptors providing both primary and costimulatory signaling in T cells from a single gene product. J Immunol 1998; 161: 2791-2797.
34 Huang X, Guo H, Kang J, Choi S, Zhou T C, Tammana S et al. Sleeping Beauty transposon-mediated engineering of human primary T cells for therapy of CD19⁺ lymphoid malignancies. Mol Ther 2008; 16: 580-589.
35 Milone M C, Fish J D, Carpenito C, Carroll R G, Binder G K, Teachey D et al. Chimeric receptors containing CD137 signal transduction domains mediate enhanced survival of T cells and increased antileukemic efficacy in vivo. Mol Ther 2009; 17:1453-1464.
36 Klebanoff C A, Finkelstein S E, Surman D R, Lichtman M K, Gattinoni L, Theoret M R et al. IL-15 enhances the in vivo antitumor activity of tumor-reactive CD8⁺ T cells. Proc Natl Acad Sci USA 2004; 101: 1969-1974.
37 Roychowdhury S, May Jr K F, Tzou K S, Lin T, Bhatt D, Freud A G et al. Failed adoptive immunotherapy with tumor-specific T cells:reversal with low-dose interleukin 15 but not low-dose interleukin 2. Cancer Res 2004; 64: 8062-8067.
38 Hsu C, Hughes M S, Zheng Z, Bray R B, Rosenberg S A, Morgan R A. Primary human T lymphocytes engineered with a codon-optimized IL-15 gene resist cytokine withdrawal-induced apoptosis and persist long-term in the absence of exogenous cytokine. J Immunol 2005; 175: 7226-7234.
39 Urbani S, Amadei B, Tola D, Massari M, Schivazappa S, Missale G et al. PD-1 expression in acute hepatitis C Virus (HCV) infection is associated with HCV-specific CD8 exhaustion. J Virol 2006; 80: 11398-11403.
40 Keir M E, Butte M J, Freeman G J, Sharpe A H. PD-1 and its ligands in tolerance and immunity. Annu Rev Immunol 2008; 26: 677-704.
41 Mumprecht S, Schurch C, Schwaller J, Solenthaler M, Ochsenbein A F. Programmed death 1 signaling on chronic myeloid leukemia specific T cells results in T-cell exhaustion and disease progression. Blood 2009; 114: 1528-1536.
42 Ahmadzadeh M, Johnson L A, Heemskerk B, Wunderlich J R, Dudley M E, White D E et al. Tumor antigen-specific CD8 T cells infiltrating the tumor express high levels of PD-1 and are functionally impaired. Blood 2009; 114: 1537-1544.
43 Zhang L, Gajewski T F, Kline J. PD-1/PD-L1 interactions inhibit antitumor immune responses in a murine acute myeloid leukemia model. Blood 2009; 114: 1545-1552.
44 Curiel T J, Wei S, Dong H, Alvarez X, Cheng P, Mottram P et al. Blockade of B7-H1 improves myeloid dendritic cell-mediated antitumor immunity. Nat Med 2003; 9: 562-567.
45 Berger C, Berger M, Hackman R C, Gough M, Elliott C, Jensen M C et al. Safety and immunologic effects of IL-15 administration in nonhuman primates. Blood 2009; 114: 2417-2426.
46 Rosenberg S A, Restifo N P, Yang J C, Morgan R A, Dudley M E. Adoptive cell transfer: a clinical path to effective cancer immunotherapy. Nat Rev Cancer 2008; 8: 299-308.
47 Dudley M E, Wunderlich J R, Yang J C, Sherry R M, Topalian S L, Restifo N P et al. Adoptive cell transfer therapy following nonmyeloablative but lymphodepleting chemotherapy for the treatment of patients with refractory metastatic melanoma. J Clin Oncol 2005; 23: 2346-2357.
48 Zou W. Immunosuppressive networks in the tumour environment and their therapeutic relevance. Nat Rev Cancer 2005; 5: 263-274.
49 Ben A M, Belhadj H N, Moes N, Buyse S, Abdeladhim M, Louzir H et al. IL-15 renders conventional lymphocytes resistant to suppressive functions of regulatory T cells through activation of the phosphatidylinositol 3-kinase pathway. J Immunol 2009; 182: 6763-6770.
50 Lamers C H, Sleijfer S, Vulto A G, Kruit W H, Kliffen M, Debets R et al. Treatment of metastatic renal cell carcinoma with autologous T-lymphocytes genetically retargeted against carbonic anhydrase IX: first clinical experience. J Clin Oncol 2006; 24: e20-e22.
51 Tey S K, Dotti G, Rooney C M, Heslop H E, Brenner M K. Inducible caspase 9 suicide gene to improve the safety of allodepleted T cells after haploidentical stem cell transplantation. Biol Blood Marrow Transplant 2007; 13: 913-924.

One skilled in the art readily appreciates that the present invention is well adapted to carry out the objectives and obtain the ends and advantages mentioned as well as those inherent therein. Methods, procedures, techniques and kits described herein are presently representative of the preferred embodiments and are intended to be exemplary and are not intended as limitations of the scope. Changes therein and other uses will occur to those skilled in the art which are encompassed within the spirit of the invention or defined by the scope of the pending claims.

What is claimed is:

1. An isolated polynucleotide, comprising a cytokine, an inducible suicide gene, and one or both of the following:
a) a detectable gene product; or
b) a chimeric antigen receptor.

2. The polynucleotide of claim 1, further defined as comprising a vector.

3. The polynucleotide of claim 2, wherein the vector is a viral vector or a plasmid.

4. The polynucleotide of claim 3, wherein the viral vector is an adenoviral vector, a retriviral vector, a lentiviral vector, or an adeno-associated viral vector.

5. The polynucleotide of claim 1, wherein the cytokine is IL-15, IL-2, IL-7, IL-12, or IL-21.

6. The polynucleotide of claim 1, wherein the inducible suicide gene is non-immunogenic to humans.

7. The polynucleotide of claim 6, wherein the inducible suicide gene is caspase 9.

8. The polynucleotide of claim 1, wherein the chimeric antigen receptor targets CD19.

9. The polynucleotide of claim 1, wherein the chimeric antigen receptor has a costimulatory endodomain from CD28, 4-IBB, OX40, or a combination thereof.

10. The polynucleotide of claim 1, wherein the detectable gene product is a nonfunctional gene product.

11. The polynucleotide of claim 1, wherein the detectable gene product is ΔNGFR, a truncated form of CD19, or a truncated form of CD34.

12. A mammalian cell, comprising the polynucleotide of claim 1.

13. The cell of claim 12, wherein the cell is a T lymphocyte, natural killer cell, lymphokine-activated killer cell, or tumor infiltrating lymphocyte.

14. A method of inhibiting proliferation of a cancer cell in an individual, comprising the step of delivering to the individual a therapeutically effective amount of cells of claim 13.

15. The method of claim 14, further defined as:
delivering to the individual a therapeutically effective amount of cells of claim 13;
releasing a relevant T cell growth factor or immunomodulating cytokine locally in the tumor microenviroment; and
eliminating the cells upon exposure to the inducible gene product.

16. A kit comprising the polynucleotide of claim 1.

17. A kit comprising one or more cells of claim 12.

18. A method of making a cell of claim 12, comprising the step of introducing to the cell a polynucleotide comprising a cytokine, an inducible suicide gene, and one or both of the following:
a) a detectable or selectable gene product; or
b) a chimeric antigen receptor.
